SEQUENCE LISTING (424 sequences)

SEQ ID NO: 1 - protein, 5 aa, Artificial Sequence
  Cyclic peptide with His-Ala-Val cell adhesion recognition motif
CHAVC

SEQ ID NO: 2 - protein, 6 aa, Artificial Sequence
  Cyclic peptide with His-Ala-Val cell adhesion recognition motif
CHAVCY

SEQ ID NO: 3 - protein, 6 aa, Artificial Sequence
  Cyclic peptide with His-Ala-Val cell adhesion recognition motif
CHAVDC

SEQ ID NO: 4 - protein, 7 aa, Artificial Sequence
  Cyclic peptide with His-Ala-Val cell adhesion recognition motif
CHAVDIC

SEQ ID NO: 5 - protein, 8 aa, Artificial Sequence
  Cyclic peptide with His-Ala-Val cell adhesion recognition motif
CHAVDINC

SEQ ID NO: 6 - protein, 9 aa, Artificial Sequence
  Cyclic peptide with His-Ala-Val cell adhesion recognition motif
CHAVDINGC

SEQ ID NO: 7 - protein, 6 aa, Artificial Sequence
  Cyclic peptide with His-Ala-Val cell adhesion recognition motif
CAHAVC

SEQ ID NO: 8 - protein, 7 aa, Artificial Sequence
  Cyclic peptide with His-Ala-Val cell adhesion recognition motif
CAHAVDC

SEQ ID NO: 9 - protein, 8 aa, Artificial Sequence
  Cyclic peptide with His-Ala-Val cell adhesion recognition motif
CAHAVDIC

SEQ ID NO: 10 - protein, 8 aa, Artificial Sequence
  Cyclic peptide with His-Ala-Val cell adhesion recognition motif
CRAHAVDC

SEQ ID NO: 11 - protein, 8 aa, Artificial Sequence
  Cyclic peptide with His-Ala-Val cell adhesion recognition motif
CLRAHAVC

SEQ ID NO: 12 - protein, 9 aa, Artificial Sequence
  Cyclic peptide with His-Ala-Val cell adhesion recognition motif
CLRAHAVDC

SEQ ID NO: 13 - protein, 6 aa, Artificial Sequence
  Cyclic peptide with His-Ala-Val cell adhesion recognition motif
CSHAVC

SEQ ID NO: 14 - protein, 7 aa, Artificial Sequence
  Cyclic peptide with His-Ala-Val cell adhesion recognition motif
CFSHAVC

SEQ ID NO: 15 - protein, 8 aa, Artificial Sequence
  Cyclic peptide with His-Ala-Val cell adhesion recognition motif
CLFSHAVC

SEQ ID NO: 16 - protein, 6 aa, Artificial Sequence
  Cyclic peptide with His-Ala-Val cell adhesion recognition motif
CHAVSC

SEQ ID NO: 17 - protein, 7 aa, Artificial Sequence
  Cyclic peptide with His-Ala-Val cell adhesion recognition motif
CSHAVSC

SEQ ID NO: 18 - protein, 8 aa, Artificial Sequence
  Cyclic peptide with His-Ala-Val cell adhesion recognition motif
CSHAVSSC

SEQ ID NO: 19 - protein, 7 aa, Artificial Sequence
  Cyclic peptide with His-Ala-Val cell adhesion recognition motif
CHAVSSC

SEQ ID NO: 20 - protein, 5 aa, Artificial Sequence
  Cyclic peptide with His-Ala-Val cell adhesion recognition motif
KHAVD

SEQ ID NO: 21 - protein, 5 aa, Artificial Sequence
  Cyclic peptide with His-Ala-Val cell adhesion recognition motif
DHAVK

SEQ ID NO: 22 - protein, 5 aa, Artificial Sequence
  Cyclic peptide with His-Ala-Val cell adhesion recognition motif
KHAVE

SEQ ID NO: 23 - protein, 6 aa, Artificial Sequence
  Cyclic peptide with His-Ala-Val cell adhesion recognition motif
AHAVDI

SEQ ID NO: 24 - protein, 7 aa, Artificial Sequence
  Cyclic peptide with His-Ala-Val cell adhesion recognition motif
SHAVDSS

SEQ ID NO: 25 - protein, 8 aa, Artificial Sequence
  Cyclic peptide with His-Ala-Val cell adhesion recognition motif
KSHAVSSD

SEQ ID NO: 26 - protein, 5 aa, Artificial Sequence
  Cyclic peptide with His-Ala-Val cell adhesion recognition motif
CHAVC

SEQ ID NO: 27 - protein, 5 aa, Artificial Sequence
  Cyclic peptide with His-Ala-Val cell adhesion recognition motif
CHAVC

SEQ ID NO: 28 - protein, 7 aa, Artificial Sequence
  Cyclic peptide with His-Ala-Val cell adhesion recognition motif
CHAVCSS

SEQ ID NO: 29 - protein, 7 aa, Artificial Sequence
  Cyclic peptide with His-Ala-Val cell adhesion recognition motif
SCHAVCS

SEQ ID NO: 30 - protein, 6 aa, Artificial Sequence
  Cyclic peptide with His-Ala-Val cell adhesion recognition motif
CHAVCT

SEQ ID NO: 31 - protein, 6 aa, Artificial Sequence
  Cyclic peptide with His-Ala-Val cell adhesion recognition motif
CHAVCE

SEQ ID NO: 32 - protein, 6 aa, Artificial Sequence
  Cyclic peptide with His-Ala-Val cell adhesion recognition motif
CHAVCD

SEQ ID NO: 33 - protein, 6 aa, Artificial Sequence
  Cyclic peptide with His-Ala-Val cell adhesion recognition motif
CHAVYC

SEQ ID NO: 34 - protein, 6 aa, Artificial Sequence
  Cyclic peptide with His-Ala-Val cell adhesion recognition motif
CHAVCY

SEQ ID NO: 35 - protein, 5 aa, Artificial Sequence
  Cyclic peptide with His-Ala-Val cell adhesion recognition motif
CHAVC

SEQ ID NO: 36 - protein, 5 aa, Artificial Sequence
  Cyclic peptide with His-Ala-Val cell adhesion recognition motif
CHAVC

SEQ ID NO: 37 - protein, 5 aa, Artificial Sequence
  Cyclic peptide with His-Ala-Val cell adhesion recognition motif
CHAVX

SEQ ID NO: 38 - protein, 5 aa, Artificial Sequence
  Cyclic peptide with His-Ala-Val cell adhesion recognition motif
XHAVC

SEQ ID NO: 39 - protein, 6 aa, Artificial Sequence
  Cyclic peptide with His-Ala-Val cell adhesion recognition motif
CHAVPC

SEQ ID NO: 40 - protein, 6 aa, Artificial Sequence
  Cadherin antagonist motif
XWVXXP

SEQ ID NO: 41 - protein, 4 aa, Artificial Sequence
  Cadherin antagonist containing Trp- cell adhesion recognition sequence
DWVI

SEQ ID NO: 42 - protein, 4 aa, Artificial Sequence
  Cadherin antagonist containing Trp- cell adhesion recognition sequence
DWVV

SEQ ID NO: 43 - protein, 4 aa, Artificial Sequence
  Cadherin antagonist containing Trp- cell adhesion recognition sequence
DWVM

SEQ ID NO: 44 - protein, 5 aa, Artificial Sequence
  Cadherin antagonist containing Trp- cell adhesion recognition sequence
DWVIP

SEQ ID NO: 45 - protein, 5 aa, Artificial Sequence
  Cadherin antagonist containing Trp- cell adhesion recognition sequence
DWVIA

SEQ ID NO: 46 - protein, 5 aa, Artificial Sequence
  Cadherin antagonist containing Trp- cell adhesion recognition sequence
DWVVP

SEQ ID NO: 47 - protein, 6 aa, Artificial Sequence
  Cadherin antagonist containing Trp- cell adhesion recognition sequence
DWVVPP

SEQ ID NO: 48 - protein, 6 aa, Artificial Sequence
  Cadherin antagonist containing Trp- cell adhesion recognition sequence
DWVVAP

SEQ ID NO: 49 - protein, 6 aa, Artificial Sequence
  Cadherin antagonist containing Trp- cell adhesion recognition sequence
DWVMPP

SEQ ID NO: 50 - protein, 6 aa, Artificial Sequence
  Cadherin antagonist containing Trp- cell adhesion recognition sequence
DWVMAP

SEQ ID NO: 51 - protein, 4 aa, Artificial Sequence
  Cadherin antagonist containing Trp- cell adhesion recognition sequence
EWVI

SEQ ID NO: 52 - protein, 4 aa, Artificial Sequence
  Cadherin antagonist containing Trp- cell adhesion recognition sequence
EWVV

SEQ ID NO: 53 - protein, 4 aa, Artificial Sequence
  Cadherin antagonist containing Trp- cell adhesion recognition sequence
EWVM

SEQ ID NO: 54 - protein, 5 aa, Artificial Sequence
  Cadherin antagonist containing Trp- cell adhesion recognition sequence
EWVIP

SEQ ID NO: 55 - protein, 5 aa, Artificial Sequence
  Cadherin antagonist containing Trp- cell adhesion recognition sequence
EWVIA

SEQ ID NO: 56 - protein, 5 aa, Artificial Sequence
  Cadherin antagonist containing Trp- cell adhesion recognition sequence
EWVVP

SEQ ID NO: 57 - protein, 6 aa, Artificial Sequence
  Cadherin antagonist containing Trp- cell adhesion recognition sequence
EWVVPP

SEQ ID NO: 58 - protein, 6 aa, Artificial Sequence
  Cadherin antagonist containing Trp- cell adhesion recognition sequence
EWVVAP

SEQ ID NO: 59 - protein, 6 aa, Artificial Sequence
  Cadherin antagonist containing Trp- cell adhesion recognition sequence
EWVMPP

SEQ ID NO: 60 - protein, 6 aa, Artificial Sequence
  Cadherin antagonist containing Trp- cell adhesion recognition sequence
EWVMAP

SEQ ID NO: 61 - protein, 7 aa, Artificial Sequence
  Cadherin antagonist containing Trp- cell adhesion recognition sequence
WVIWVIP

SEQ ID NO: 62 - protein, 4 aa, Artificial Sequence
  Cadherin antagonist containing Trp- cell adhesion recognition sequence
WVIA

SEQ ID NO: 63 - protein, 4 aa, Artificial Sequence
  Cadherin antagonist containing Trp- cell adhesion recognition sequence
WVVP

SEQ ID NO: 64 - protein, 4 aa, Artificial Sequence
  Cadherin antagonist containing Trp- cell adhesion recognition sequence
WVVA

SEQ ID NO: 65 - protein, 4 aa, Artificial Sequence
  Cadherin antagonist containing Trp- cell adhesion recognition sequence
WVMP

SEQ ID NO: 66 - protein, 4 aa, Artificial Sequence
  Cadherin antagonist containing Trp- cell adhesion recognition sequence
WVMA

SEQ ID NO: 67 - protein, 5 aa, Artificial Sequence
  Cadherin antagonist containing Trp- cell adhesion recognition sequence
WVIPP

SEQ ID NO: 68 - protein, 5 aa, Artificial Sequence
  Cadherin antagonist containing Trp- cell adhesion recognition sequence
WVIAP

SEQ ID NO: 69 - protein, 5 aa, Artificial Sequence
  Cadherin antagonist containing Trp- cell adhesion recognition sequence
WVVPP

SEQ ID NO: 70 - protein, 5 aa, Artificial Sequence
  Cadherin antagonist containing Trp- cell adhesion recognition sequence
WVVAP

SEQ ID NO: 71 - protein, 5 aa, Artificial Sequence
  Cadherin antagonist containing Trp- cell adhesion recognition sequence
WVMPP

SEQ ID NO: 72 - protein, 5 aa, Artificial Sequence
  Cadherin antagonist containing Trp- cell adhesion recognition sequence
WVMAP

SEQ ID NO: 73 - protein, 4 aa, Artificial Sequence
  Cadherin antagonist containing Trp- cell adhesion recognition sequence
DWII

SEQ ID NO: 74 - protein, 4 aa, Artificial Sequence
  Cadherin antagonist containing Trp- cell adhesion recognition sequence
DWIV

SEQ ID NO: 75 - protein, 4 aa, Artificial Sequence
  Cadherin antagonist containing Trp- cell adhesion recognition sequence
DWIM

SEQ ID NO: 76 - protein, 5 aa, Artificial Sequence
  Cadherin antagonist containing Trp- cell adhesion recognition sequence
DWIIP

SEQ ID NO: 77 - protein, 5 aa, Artificial Sequence
  Cadherin antagonist containing Trp- cell adhesion recognition sequence
DWIIA

SEQ ID NO: 78 - protein, 5 aa, Artificial Sequence
  Cadherin antagonist containing Trp- cell adhesion recognition sequence
DWIVP

SEQ ID NO: 79 - protein, 6 aa, Artificial Sequence
  Cadherin antagonist containing Trp- cell adhesion recognition sequence
DWIVPP

SEQ ID NO: 80 - protein, 6 aa, Artificial Sequence
  Cadherin antagonist containing Trp- cell adhesion recognition sequence
DWIVAP

SEQ ID NO: 81 - protein, 6 aa, Artificial Sequence
  Cadherin antagonist containing Trp- cell adhesion recognition sequence
DWIMPP

SEQ ID NO: 82 - protein, 6 aa, Artificial Sequence
  Cadherin antagonist containing Trp- cell adhesion recognition sequence
DWIMAP

SEQ ID NO: 83 - protein, 4 aa, Artificial Sequence
  Cadherin antagonist containing Trp- cell adhesion recognition sequence
EWII

SEQ ID NO: 84 - protein, 4 aa, Artificial Sequence
  Cadherin antagonist containing Trp- cell adhesion recognition sequence
EWIV

SEQ ID NO: 85 - protein, 4 aa, Artificial Sequence
  Cadherin antagonist containing Trp- cell adhesion recognition sequence
EWIM

SEQ ID NO: 86 - protein, 5 aa, Artificial Sequence
  Cadherin antagonist containing Trp- cell adhesion recognition sequence
EWIIP

SEQ ID NO: 87 - protein, 5 aa, Artificial Sequence
  Cadherin antagonist containing Trp- cell adhesion recognition sequence
EWIIA

SEQ ID NO: 88 - protein, 5 aa, Artificial Sequence
  Cadherin antagonist containing Trp- cell adhesion recognition sequence
EWIVP

SEQ ID NO: 89 - protein, 6 aa, Artificial Sequence
  Cadherin antagonist containing Trp- cell adhesion recognition sequence
EWIVPP

SEQ ID NO: 90 - protein, 6 aa, Artificial Sequence
  Cadherin antagonist containing Trp- cell adhesion recognition sequence
EWIVAP

SEQ ID NO: 91 - protein, 6 aa, Artificial Sequence
  Cadherin antagonist containing Trp- cell adhesion recognition sequence
EWIMPP

SEQ ID NO: 92 - protein, 6 aa, Artificial Sequence
  Cadherin antagonist containing Trp- cell adhesion recognition sequence
EWIMAP

SEQ ID NO: 93 - protein, 4 aa, Artificial Sequence
  Cadherin antagonist containing Trp- cell adhesion recognition sequence
WIIP

SEQ ID NO: 94 - protein, 4 aa, Artificial Sequence
  Cadherin antagonist containing Trp- cell adhesion recognition sequence
WIIA

SEQ ID NO: 95 - protein, 4 aa, Artificial Sequence
  Cadherin antagonist containing Trp- cell adhesion recognition sequence
WIVP

SEQ ID NO: 96 - protein, 4 aa, Artificial Sequence
  Cadherin antagonist containing Trp- cell adhesion recognition sequence
WIVA

SEQ ID NO: 97 - protein, 4 aa, Artificial Sequence
  Cadherin antagonist containing Trp- cell adhesion recognition sequence
WIMP

SEQ ID NO: 98 - protein, 4 aa, Artificial Sequence
  Cadherin antagonist containing Trp- cell adhesion recognition sequence
WIMA

SEQ ID NO: 99 - protein, 5 aa, Artificial Sequence
  Cadherin antagonist containing Trp- cell adhesion recognition sequence
WIIPP

SEQ ID NO: 100 - protein, 5 aa, Artificial Sequence
  Cadherin antagonist containing Trp- cell adhesion recognition sequence
WIIAP

SEQ ID NO: 101 - protein, 5 aa, Artificial Sequence
  Cadherin antagonist containing Trp- cell adhesion recognition sequence
WIVPP

SEQ ID NO: 102 - protein, 5 aa, Artificial Sequence
  Cadherin antagonist containing Trp- cell adhesion recognition sequence
WIVAP

SEQ ID NO: 103 - protein, 5 aa, Artificial Sequence
  Cadherin antagonist containing Trp- cell adhesion recognition sequence
WIMPP

SEQ ID NO: 104 - protein, 5 aa, Artificial Sequence
  Cadherin antagonist containing Trp- cell adhesion recognition sequence
WIMAP

SEQ ID NO: 105 - protein, 4 aa, Artificial Sequence
  Cadherin antagonist containing Trp- cell adhesion recognition sequence
DWLI

SEQ ID NO: 106 - protein, 4 aa, Artificial Sequence
  Cadherin antagonist containing Trp- cell adhesion recognition sequence
DWLV

SEQ ID NO: 107 - protein, 4 aa, Artificial Sequence
  Cadherin antagonist containing Trp- cell adhesion recognition sequence
DWLM

SEQ ID NO: 108 - protein, 5 aa, Artificial Sequence
  Cadherin antagonist containing Trp- cell adhesion recognition sequence
DWLIP

SEQ ID NO: 109 - protein, 5 aa, Artificial Sequence
  Cadherin antagonist containing Trp- cell adhesion recognition sequence
DWLIA

SEQ ID NO: 110 - protein, 5 aa, Artificial Sequence
  Cadherin antagonist containing Trp- cell adhesion recognition sequence
DWLVP

SEQ ID NO: 111 - protein, 6 aa, Artificial Sequence
  Cadherin antagonist containing Trp- cell adhesion recognition sequence
DWLVPP

SEQ ID NO: 112 - protein, 6 aa, Artificial Sequence
  Cadherin antagonist containing Trp- cell adhesion recognition sequence
DWLVAP

SEQ ID NO: 113 - protein, 6 aa, Artificial Sequence
  Cadherin antagonist containing Trp- cell adhesion recognition sequence
DWLMPP

SEQ ID NO: 114 - protein, 6 aa, Artificial Sequence
  Cadherin antagonist containing Trp- cell adhesion recognition sequence
DWLMAP

SEQ ID NO: 115 - protein, 4 aa, Artificial Sequence
  Cadherin antagonist containing Trp- cell adhesion recognition sequence
EWLI

SEQ ID NO: 116 - protein, 4 aa, Artificial Sequence
  Cadherin antagonist containing Trp- cell adhesion recognition sequence
EWLV

SEQ ID NO: 117 - protein, 4 aa, Artificial Sequence
  Cadherin antagonist containing Trp- cell adhesion recognition sequence
EWLM

SEQ ID NO: 118 - protein, 5 aa, Artificial Sequence
  Cadherin antagonist containing Trp- cell adhesion recognition sequence
EWLIP

SEQ ID NO: 119 - protein, 5 aa, Artificial Sequence
  Cadherin antagonist containing Trp- cell adhesion recognition sequence
EWLIA

SEQ ID NO: 120 - protein, 5 aa, Artificial Sequence
  Cadherin antagonist containing Trp- cell adhesion recognition sequence
EWLVP

SEQ ID NO: 121 - protein, 6 aa, Artificial Sequence
  Cadherin antagonist containing Trp- cell adhesion recognition sequence
EWLVPP

SEQ ID NO: 122 - protein, 6 aa, Artificial Sequence
  Cadherin antagonist containing Trp- cell adhesion recognition sequence
EWLVAP

SEQ ID NO: 123 - protein, 6 aa, Artificial Sequence
  Cadherin antagonist containing Trp- cell adhesion recognition sequence
EWLMPP

SEQ ID NO: 124 - protein, 6 aa, Artificial Sequence
  Cadherin antagonist containing Trp- cell adhesion recognition sequence
EWLMAP

SEQ ID NO: 125 - protein, 4 aa, Artificial Sequence
  Cadherin antagonist containing Trp- cell adhesion recognition sequence
WLIP

SEQ ID NO: 126 - protein, 4 aa, Artificial Sequence
  Cadherin antagonist containing Trp- cell adhesion recognition sequence
WLIA

SEQ ID NO: 127 - protein, 7 aa, Artificial Sequence
  Cadherin antagonist containing Trp- cell adhesion recognition sequence
WLVWLVP

SEQ ID NO: 128 - protein, 4 aa, Artificial Sequence
  Cadherin antagonist containing Trp- cell adhesion recognition sequence
WLVA

SEQ ID NO: 129 - protein, 4 aa, Artificial Sequence
  Cadherin antagonist containing Trp- cell adhesion recognition sequence
WLMP

SEQ ID NO: 130 - protein, 4 aa, Artificial Sequence
  Cadherin antagonist containing Trp- cell adhesion recognition sequence
WLMA

SEQ ID NO: 131 - protein, 5 aa, Artificial Sequence
  Cadherin antagonist containing Trp- cell adhesion recognition sequence
WLIPP

SEQ ID NO: 132 - protein, 5 aa, Artificial Sequence
  Cadherin antagonist containing Trp- cell adhesion recognition sequence
WLIAP

SEQ ID NO: 133 - protein, 5 aa, Artificial Sequence
  Cadherin antagonist containing Trp- cell adhesion recognition sequence
WLVPP

SEQ ID NO: 134 - protein, 5 aa, Artificial Sequence
  Cadherin antagonist containing Trp- cell adhesion recognition sequence
WLVAP

SEQ ID NO: 135 - protein, 5 aa, Artificial Sequence
  Cadherin antagonist containing Trp- cell adhesion recognition sequence
WLMPP

SEQ ID NO: 136 - protein, 5 aa, Artificial Sequence
  Cadherin antagonist containing Trp- cell adhesion recognition sequence
WLMAP

SEQ ID NO: 137 - protein, 4 aa, Artificial Sequence
  Cadherin antagonist containing Trp- cell adhesion recognition sequence
DWVL

SEQ ID NO: 138 - protein, 4 aa, Artificial Sequence
  Cadherin antagonist containing Trp- cell adhesion recognition sequence
DWIL

SEQ ID NO: 139 - protein, 4 aa, Artificial Sequence
  Cadherin antagonist containing Trp- cell adhesion recognition sequence
DWLL

SEQ ID NO: 140 - protein, 4 aa, Artificial Sequence
  Cadherin antagonist containing Trp- cell adhesion recognition sequence
EWVL

SEQ ID NO: 141 - protein, 4 aa, Artificial Sequence
  Cadherin antagonist containing Trp- cell adhesion recognition sequence
EWIL

SEQ ID NO: 142 - protein, 4 aa, Artificial Sequence
  Cadherin antagonist containing Trp- cell adhesion recognition sequence
EWLL

SEQ ID NO: 143 - protein, 5 aa, Artificial Sequence
  Cadherin antagonist containing Trp- cell adhesion recognition sequence
DWVLP

SEQ ID NO: 144 - protein, 5 aa, Artificial Sequence
  Cadherin antagonist containing Trp- cell adhesion recognition sequence
DWILP

SEQ ID NO: 145 - protein, 5 aa, Artificial Sequence
  Cadherin antagonist containing Trp- cell adhesion recognition sequence
DWLLP

SEQ ID NO: 146 - protein, 5 aa, Artificial Sequence
  Cadherin antagonist containing Trp- cell adhesion recognition sequence
EWVLP

SEQ ID NO: 147 - protein, 5 aa, Artificial Sequence
  Cadherin antagonist containing Trp- cell adhesion recognition sequence
EWILP

SEQ ID NO: 148 - protein, 5 aa, Artificial Sequence
  Cadherin antagonist containing Trp- cell adhesion recognition sequence
EWLLP

SEQ ID NO: 149 - protein, 5 aa, Artificial Sequence
  Cadherin antagonist containing Trp- cell adhesion recognition sequence
DWVLA

SEQ ID NO: 150 - protein, 5 aa, Artificial Sequence
  Cadherin antagonist containing Trp- cell adhesion recognition sequence
DWILA

SEQ ID NO: 151 - protein, 5 aa, Artificial Sequence
  Cadherin antagonist containing Trp- cell adhesion recognition sequence
DWLLA

SEQ ID NO: 152 - protein, 5 aa, Artificial Sequence
  Cadherin antagonist containing Trp- cell adhesion recognition sequence
EWVLA

SEQ ID NO: 153 - protein, 5 aa, Artificial Sequence
  Cadherin antagonist containing Trp- cell adhesion recognition sequence
EWILA

SEQ ID NO: 154 - protein, 5 aa, Artificial Sequence
  Cadherin antagonist containing Trp- cell adhesion recognition sequence
EWLLA

SEQ ID NO: 155 - protein, 6 aa, Artificial Sequence
  Cadherin antagonist containing Trp- cell adhesion recognition sequence
DWVLPP

SEQ ID NO: 156 - protein, 6 aa, Artificial Sequence
  Cadherin antagonist containing Trp- cell adhesion recognition sequence
DWILPP

SEQ ID NO: 157 - protein, 6 aa, Artificial Sequence
  Cadherin antagonist containing Trp- cell adhesion recognition sequence
DWLLPP

SEQ ID NO: 158 - protein, 6 aa, Artificial Sequence
  Cadherin antagonist containing Trp- cell adhesion recognition sequence
EWVLPP

SEQ ID NO: 159 - protein, 6 aa, Artificial Sequence
  Cadherin antagonist containing Trp- cell adhesion recognition sequence
EWILPP

SEQ ID NO: 160 - protein, 6 aa, Artificial Sequence
  Cadherin antagonist containing Trp- cell adhesion recognition sequence
EWLLPP

SEQ ID NO: 161 - protein, 6 aa, Artificial Sequence
  Cadherin antagonist containing Trp- cell adhesion recognition sequence
DWVLAP

SEQ ID NO: 162 - protein, 6 aa, Artificial Sequence
  Cadherin antagonist containing Trp- cell adhesion recognition sequence
DWILAP

SEQ ID NO: 163 - protein, 6 aa, Artificial Sequence
  Cadherin antagonist containing Trp- cell adhesion recognition sequence
DWLLAP

SEQ ID NO: 164 - protein, 6 aa, Artificial Sequence
  Cadherin antagonist containing Trp- cell adhesion recognition sequence
EWVLAP

SEQ ID NO: 165 - protein, 6 aa, Artificial Sequence
  Cadherin antagonist containing Trp- cell adhesion recognition sequence
EWILAP

SEQ ID NO: 166 - protein, 6 aa, Artificial Sequence
  Cadherin antagonist containing Trp- cell adhesion recognition sequence
EWLLAP

SEQ ID NO: 167 - protein, 4 aa, Artificial Sequence
  Cadherin antagonist containing Trp- cell adhesion recognition sequence
WVLP

SEQ ID NO: 168 - protein, 4 aa, Artificial Sequence
  Cadherin antagonist containing Trp- cell adhesion recognition sequence
WILP

SEQ ID NO: 169 - protein, 4 aa, Artificial Sequence
  Cadherin antagonist containing Trp- cell adhesion recognition sequence
WLLP

SEQ ID NO: 170 - protein, 4 aa, Artificial Sequence
  Cadherin antagonist containing Trp- cell adhesion recognition sequence
WVLA

SEQ ID NO: 171 - protein, 4 aa, Artificial Sequence
  Cadherin antagonist containing Trp- cell adhesion recognition sequence
WILA

SEQ ID NO: 172 - protein, 4 aa, Artificial Sequence
  Cadherin antagonist containing Trp- cell adhesion recognition sequence
WLLA

SEQ ID NO: 173 - protein, 5 aa, Artificial Sequence
  Cadherin antagonist containing Trp- cell adhesion recognition sequence
WVLPP

SEQ ID NO: 174 - protein, 5 aa, Artificial Sequence
  Cadherin antagonist containing Trp- cell adhesion recognition sequence
WILPP

SEQ ID NO: 175 - protein, 5 aa, Artificial Sequence
  Cadherin antagonist containing Trp- cell adhesion recognition sequence
WLLPP

SEQ ID NO: 176 - protein, 5 aa, Artificial Sequence
  Cadherin antagonist containing Trp- cell adhesion recognition sequence
WVLAP

SEQ ID NO: 177 - protein, 5 aa, Artificial Sequence
  Cadherin antagonist containing Trp- cell adhesion recognition sequence
WILAP

SEQ ID NO: 178 - protein, 5 aa, Artificial Sequence
  Cadherin antagonist containing Trp- cell adhesion recognition sequence
WLLAP

SEQ ID NO: 179 - protein, 6 aa, Artificial Sequence
  Cadherin antagonist containing Trp- cell adhesion recognition sequence
DWVIPP

SEQ ID NO: 180 - protein, 5 aa, Artificial Sequence
  Cadherin antagonist containing Trp- cell adhesion recognition sequence
DWVVA

SEQ ID NO: 181 - protein, 5 aa, Artificial Sequence
  Cadherin antagonist containing Trp- cell adhesion recognition sequence
EWVMP

SEQ ID NO: 182 - protein, 11 aa, Artificial Sequence
  Cadherin antagonist containing HAV binding motif cell adhesion recognition sequence
XFXIXXXXGXX

SEQ ID NO: 183 - protein, 11 aa, Artificial Sequence
  Cadherin antagonist containing HAV binding motif cell adhesion recognition sequence
WLXIXXXXGQI

SEQ ID NO: 184 - protein, 11 aa, Artificial Sequence
  Cadherin antagonist containing HAV binding motif
IFIINPISGQL

SEQ ID NO: 185 - protein, 11 aa, Artificial Sequence
  Cadherin antagonist containing HAV binding motif
IFILNPISGQL

SEQ ID NO: 186 - protein, 11 aa, Artificial Sequence
  Cadherin antagonist containing HAV binding motif
VFAVEKETGWL

SEQ ID NO: 187 - protein, 11 aa, Artificial Sequence
  Cadherin antagonist containing HAV binding motif
VFSINSMSGRM

SEQ ID NO: 188 - protein, 11 aa, Artificial Sequence
  Cadherin antagonist containing HAV binding motif
VFIIERETGWL

SEQ ID NO: 189 - protein, 11 aa, Artificial Sequence
  Cadherin antagonist containing HAV binding motif
VFTIEKESGWL

SEQ ID NO: 190 - protein, 11 aa, Artificial Sequence
  Cadherin antagonist containing HAV binding motif
VFNIDSMSGRM

SEQ ID NO: 191 - protein, 11 aa, Artificial Sequence
  Cadherin antagonist containing HAV binding motif
WLKIDSVNGQI

SEQ ID NO: 192 - protein, 11 aa, Artificial Sequence
  Cadherin antagonist containing HAV binding motif
WLKIDPVNGQI

SEQ ID NO: 193 - protein, 11 aa, Artificial Sequence
  Cadherin antagonist containing HAV binding motif
WLAMDPDSGQV

SEQ ID NO: 194 - protein, 11 aa, Artificial Sequence
  Cadherin antagonist containing HAV binding motif
WLHINATNGQI

SEQ ID NO: 195 - protein, 11 aa, Artificial Sequence
  Cadherin antagonist containing HAV binding motif
WLEINPDTGAI

SEQ ID NO: 196 - protein, 11 aa, Artificial Sequence
  Cadherin antagonist containing HAV binding motif
WLAVDPDSGQI

SEQ ID NO: 197 - protein, 11 aa, Artificial Sequence
  Cadherin antagonist containing HAV binding motif
WLEINPETGAI

SEQ ID NO: 198 - protein, 11 aa, Artificial Sequence
  Cadherin antagonist containing HAV binding motif
WLHINTSNGQI

SEQ ID NO: 199 - protein, 11 aa, Artificial Sequence
  Cadherin antagonist containing HAV binding motif
NLKIDPVNGQI

SEQ ID NO: 200 - protein, 10 aa, Artificial Sequence
  Cadherin antagonist containing HAV binding motif
LKIDPVNGQI

SEQ ID NO: 201 - protein, 7 aa, Artificial Sequence
  Cadherin antagonist containing HAV binding motif
INPISGQ

SEQ ID NO: 202 - protein, 7 aa, Artificial Sequence
  Cadherin antagonist containing HAV binding motif
LNPISGQ

SEQ ID NO: 203 - protein, 7 aa, Artificial Sequence
  Cadherin antagonist containing HAV binding motif
IDPVSGQ

SEQ ID NO: 204 - protein, 8 aa, Artificial Sequence
  Cadherin antagonist containing HAV binding motif
KIDPVNGQ

SEQ ID NO: 205 - protein, 5 aa, Artificial Sequence
  Cadherin antagonist containing HAV binding motif
PISGQ

SEQ ID NO: 206 - protein, 5 aa, Artificial Sequence
  Cadherin antagonist containing HAV binding motif
PVNGQ

SEQ ID NO: 207 - protein, 5 aa, Artificial Sequence
  Cadherin antagonist containing HAV binding motif
PVSGR

SEQ ID NO: 208 - protein, 5 aa, Artificial Sequence
  Cadherin antagonist containing HAV binding motif
IDPVN

SEQ ID NO: 209 - protein, 5 aa, Artificial Sequence
  Cadherin antagonist containing HAV binding motif
INPIS

SEQ ID NO: 210 - protein, 5 aa, Artificial Sequence
  Cadherin antagonist containing HAV binding motif
KIDPV

SEQ ID NO: 211 - protein, 9 aa, Artificial Sequence
  non-classical cadherin cell adhesion recognition sequence
XFXXXXXXG

SEQ ID NO: 212 - protein, 4 aa, Artificial Sequence
  OB-cadherin antagonist
IDDK

SEQ ID NO: 213 - protein, 4 aa, Artificial Sequence
  OB-cadherin antagonist
DDKS

SEQ ID NO: 214 - protein, 5 aa, Artificial Sequence
  OB-cadherin antagonist
VIDDK

SEQ ID NO: 215 - protein, 5 aa, Artificial Sequence
  OB-cadherin antagonist
IDDKS

SEQ ID NO: 216 - protein, 6 aa, Artificial Sequence
  OB-cadherin antagonist
VIDDKS

SEQ ID NO: 217 - protein, 5 aa, Artificial Sequence
  OB-cadherin antagonist
DDKSG

SEQ ID NO: 218 - protein, 6 aa, Artificial Sequence
  OB-cadherin antagonist
IDDKSG

SEQ ID NO: 219 - protein, 7 aa, Artificial Sequence
  OB-cadherin antagonist
VIDDKSG

SEQ ID NO: 220 - protein, 6 aa, Artificial Sequence
  OB-cadherin antagonist
FVIDDK

SEQ ID NO: 221 - protein, 7 aa, Artificial Sequence
  OB-cadherin antagonist
FVIDDKS

SEQ ID NO: 222 - protein, 8 aa, Artificial Sequence
  OB-cadherin antagonist
FVIDDKSG

SEQ ID NO: 223 - protein, 7 aa, Artificial Sequence
  OB-cadherin antagonist
IFVIDDK

SEQ ID NO: 224 - protein, 8 aa, Artificial Sequence
  OB-cadherin antagonist
IFVIDDKS

SEQ ID NO: 225 - protein, 9 aa, Artificial Sequence
  OB-cadherin antagonist
IFVIDDKSG

SEQ ID NO: 226 - protein, 4 aa, Artificial Sequence
  OB-cadherin antagonist
IEEY

SEQ ID NO: 227 - protein, 4 aa, Artificial Sequence
  OB-cadherin antagonist
EEYT

SEQ ID NO: 228 - protein, 5 aa, Artificial Sequence
  OB-cadherin antagonist
VIEEY

SEQ ID NO: 229 - protein, 5 aa, Artificial Sequence
  OB-cadherin antagonist
IEEYT

SEQ ID NO: 230 - protein, 6 aa, Artificial Sequence
  OB-cadherin antagonist
VIEEYT

SEQ ID NO: 231 - protein, 5 aa, Artificial Sequence
  OB-cadherin antagonist
EEYTG

SEQ ID NO: 232 - protein, 6 aa, Artificial Sequence
  OB-cadherin antagonist
IEEYTG

SEQ ID NO: 233 - protein, 7 aa, Artificial Sequence
  OB-cadherin antagonist
VIEEYTG

SEQ ID NO: 234 - protein, 6 aa, Artificial Sequence
  OB-cadherin antagonist
FVIEEY

SEQ ID NO: 235 - protein, 7 aa, Artificial Sequence
  OB-cadherin antagonist
FVIEEYT

SEQ ID NO: 236 - protein, 8 aa, Artificial Sequence
  OB-cadherin antagonist
FVIEEYTG

SEQ ID NO: 237 - protein, 7 aa, Artificial Sequence
  OB-cadherin antagonist
FFVIEEY

SEQ ID NO: 238 - protein, 8 aa, Artificial Sequence
  OB-cadherin antagonist
FFVIEEYT

SEQ ID NO: 239 - protein, 9 aa, Artificial Sequence
  OB-cadherin antagonist
FFVIEEYTG

SEQ ID NO: 240 - protein, 4 aa, Artificial Sequence
  OB-cadherin antagonist
VEAQ

SEQ ID NO: 241 - protein, 4 aa, Artificial Sequence
  OB-cadherin antagonist
EAQT

SEQ ID NO: 242 - protein, 5 aa, Artificial Sequence
  OB-cadherin antagonist
SVEAQ

SEQ ID NO: 243 - protein, 5 aa, Artificial Sequence
  OB-cadherin antagonist
VEAQT

SEQ ID NO: 244 - protein, 6 aa, Artificial Sequence
  OB-cadherin antagonist
SVEAQT

SEQ ID NO: 245 - protein, 5 aa, Artificial Sequence
  OB-cadherin antagonist
EAQTG

SEQ ID NO: 246 - protein, 6 aa, Artificial Sequence
  OB-cadherin antagonist
VEAQTG

SEQ ID NO: 247 - protein, 7 aa, Artificial Sequence
  OB-cadherin antagonist
SVEAQTG

SEQ ID NO: 248 - protein, 6 aa, Artificial Sequence
  OB-cadherin antagonist
FSVEAQ

SEQ ID NO: 249 - protein, 7 aa, Artificial Sequence
  OB-cadherin antagonist
FSVEAQT

SEQ ID NO: 250 - protein, 8 aa, Artificial Sequence
  OB-cadherin antagonist
FSVEAQTG

SEQ ID NO: 251 - protein, 7 aa, Artificial Sequence
  OB-cadherin antagonist
YFSVEAQ

SEQ ID NO: 252 - protein, 8 aa, Artificial Sequence
  OB-cadherin antagonist
YFSVEAQT

SEQ ID NO: 253 - protein, 9 aa, Artificial Sequence
  OB-cadherin antagonist
YFSVEAQTG

SEQ ID NO: 254 - protein, 4 aa, Artificial Sequence
  VE-cadherin antagonist
VDAE

SEQ ID NO: 255 - protein, 4 aa, Artificial Sequence
  VE-cadherin antagonist
DAET

SEQ ID NO: 256 - protein, 5 aa, Artificial Sequence
  VE-cadherin antagonist
RVDAE

SEQ ID NO: 257 - protein, 5 aa, Artificial Sequence
  VE-cadherin antagonist
VDAET

SEQ ID NO: 258 - protein, 6 aa, Artificial Sequence
  VE-cadherin antagonist
RVDAET

SEQ ID NO: 259 - protein, 5 aa, Artificial Sequence
  VE-cadherin antagonist
DAETG

SEQ ID NO: 260 - protein, 6 aa, Artificial Sequence
  VE-cadherin antagonist
VDAETG

SEQ ID NO: 261 - protein, 7 aa, Artificial Sequence
  VE-cadherin antagonist
RVDAETG

SEQ ID NO: 262 - protein, 6 aa, Artificial Sequence
  VE-cadherin antagonist
FRVDAE

SEQ ID NO: 263 - protein, 7 aa, Artificial Sequence
  VE-cadherin antagonist
FRVDAET

SEQ ID NO: 264 - protein, 8 aa, Artificial Sequence
  VE-cadherin antagonist
FRVDAETG

SEQ ID NO: 265 - protein, 7 aa, Artificial Sequence
  VE-cadherin antagonist
VFRVDAE

SEQ ID NO: 266 - protein, 8 aa, Artificial Sequence
  VE-cadherin antagonist
VFRVDAET

SEQ ID NO: 267 - protein, 9 aa, Artificial Sequence
  VE-cadherin antagonist
VFRVDAETG

SEQ ID NO: 268 - protein, 6 aa, Artificial Sequence
  Atypical cadherin Trp-containing cell adhesion recognition sequence
XWXWNQ

SEQ ID NO: 269 - protein, 4 aa, Artificial Sequence
  Atypical cadherin Trp-containing cell adhesion recognition sequence
GWVW

SEQ ID NO: 270 - protein, 5 aa, Artificial Sequence
  Atypical cadherin Trp-containing cell adhesion recognition sequence
GWVWN

SEQ ID NO: 271 - protein, 6 aa, Artificial Sequence
  Atypical cadherin Trp-containing cell adhesion recognition sequence
GWVWNQ

SEQ ID NO: 272 - protein, 4 aa, Artificial Sequence
  Atypical cadherin Trp-containing cell adhesion recognition sequence
WVWN

SEQ ID NO: 273 - protein, 5 aa, Artificial Sequence
  Atypical cadherin Trp-containing cell adhesion recognition sequence
WVWNQ

SEQ ID NO: 274 - protein, 4 aa, Artificial Sequence
  Atypical cadherin Trp-containing cell adhesion recognition sequence
DWIW

SEQ ID NO: 275 - protein, 5 aa, Artificial Sequence
  Atypical cadherin Trp-containing cell adhesion recognition sequence
DWIWN

SEQ ID NO: 276 - protein, 6 aa, Artificial Sequence
  Atypical cadherin Trp-containing cell adhesion recognition sequence
DWIWNQ

SEQ ID NO: 277 - protein, 4 aa, Artificial Sequence
  Atypical cadherin Trp-containing cell adhesion recognition sequence
WIWN

SEQ ID NO: 278 - protein, 5 aa, Artificial Sequence
  Atypical cadherin Trp-containing cell adhesion recognition sequence
WIWNQ

SEQ ID NO: 279 - protein, 7 aa, Artificial Sequence
  Atypical cadherin Trp-containing cell adhesion recognition sequence
SWMSWMW

SEQ ID NO: 280 - protein, 5 aa, Artificial Sequence
  Atypical cadherin Trp-containing cell adhesion recognition sequence
SWMWN

SEQ ID NO: 281 - protein, 6 aa, Artificial Sequence
  Atypical cadherin Trp-containing cell adhesion recognition sequence
SWMWNQ

SEQ ID NO: 282 - protein, 4 aa, Artificial Sequence
  Atypical cadherin Trp-containing cell adhesion recognition sequence
WMWN

SEQ ID NO: 283 - protein, 5 aa, Artificial Sequence
  Atypical cadherin Trp-containing cell adhesion recognition sequence
WMWNQ

SEQ ID NO: 284 - protein, 4 aa, Artificial Sequence
  Atypical cadherin Trp-containing cell adhesion recognition sequence
SWVW

SEQ ID NO: 285 - protein, 5 aa, Artificial Sequence
  Atypical cadherin Trp-containing cell adhesion recognition sequence
SWVWN

SEQ ID NO: 286 - protein, 6 aa, Artificial Sequence
  Atypical cadherin Trp-containing cell adhesion recognition sequence
SWVWNQ

SEQ ID NO: 287 - protein, 4 aa, Artificial Sequence
  Atypical cadherin Trp-containing cell adhesion recognition sequence
GWMW

SEQ ID NO: 288 - protein, 5 aa, Artificial Sequence
  Atypical cadherin Trp-containing cell adhesion recognition sequence
GWMWN

SEQ ID NO: 289 - protein, 6 aa, Artificial Sequence
  Atypical cadherin Trp-containing cell adhesion recognition sequence
GWMWNQ

SEQ ID NO: 290 - protein, 4 aa, Artificial Sequence
  Atypical cadherin Trp-containing cell adhesion recognition sequence
AWVI

SEQ ID NO: 291 - protein, 5 aa, Artificial Sequence
  Atypical cadherin Trp-containing cell adhesion recognition sequence
AWVIP

SEQ ID NO: 292 - protein, 6 aa, Artificial Sequence
  Atypical cadherin Trp-containing cell adhesion recognition sequence
AWVIPP

SEQ ID NO: 293 - protein, 4 aa, Artificial Sequence
  Atypical cadherin Trp-containing cell adhesion recognition sequence
WVIP

SEQ ID NO: 294 - protein, 5 aa, Artificial Sequence
  Atypical cadherin Trp-containing cell adhesion recognition sequence
WVIPP

SEQ ID NO: 295 - protein, 7 aa, Artificial Sequence
  Atypical cadherin Trp-containing cell adhesion recognition sequence
GWVWNQF

SEQ ID NO: 296 - protein, 8 aa, Artificial Sequence
  Atypical cadherin Trp-containing cell adhesion recognition sequence
GWVWNQFF

SEQ ID NO: 297 - protein, 9 aa, Artificial Sequence
  Atypical cadherin Trp-containing cell adhesion recognition sequence
GWVWNQFFV

SEQ ID NO: 298 - protein, 6 aa, Artificial Sequence
  Atypical cadherin Trp-containing cell adhesion recognition sequence
WVWNQF

SEQ ID NO: 299 - protein, 7 aa, Artificial Sequence
  Atypical cadherin Trp-containing cell adhesion recognition sequence
WVWNQFF

SEQ ID NO: 300 - protein, 8 aa, Artificial Sequence
  Atypical cadherin Trp-containing cell adhesion recognition sequence
WVWNQFFV

SEQ ID NO: 301 - protein, 4 aa, Artificial Sequence
  Atypical cadherin Trp-containing cell adhesion recognition sequence
RGWV

SEQ ID NO: 302 - protein, 5 aa, Artificial Sequence
  Atypical cadherin Trp-containing cell adhesion recognition sequence
RGWVW

SEQ ID NO: 303 - protein, 6 aa, Artificial Sequence
  Atypical cadherin Trp-containing cell adhesion recognition sequence
RGWVWN

SEQ ID NO: 304 - protein, 7 aa, Artificial Sequence
  Atypical cadherin Trp-containing cell adhesion recognition sequence
RGWVWNQ

SEQ ID NO: 305 - protein, 8 aa, Artificial Sequence
  Atypical cadherin Trp-containing cell adhesion recognition sequence
RGWVWNQF

SEQ ID NO: 306 - protein, 9 aa, Artificial Sequence
  Atypical cadherin Trp-containing cell adhesion recognition sequence
RGWVWNQFF

SEQ ID NO: 307 - protein, 10 aa, Artificial Sequence
  Atypical cadherin Trp-containing cell adhesion recognition sequence
RGWVWNQFFV

SEQ ID NO: 308 - protein, 4 aa, Artificial Sequence
  Atypical cadherin Trp-containing cell adhesion recognition sequence
KRGW

SEQ ID NO: 309 - protein, 5 aa, Artificial Sequence
  Atypical cadherin Trp-containing cell adhesion recognition sequence
KRGWV

SEQ ID NO: 310 - protein, 6 aa, Artificial Sequence
  Atypical cadherin Trp-containing cell adhesion recognition sequence
KRGWVW

SEQ ID NO: 311 - protein, 7 aa, Artificial Sequence
  Atypical cadherin Trp-containing cell adhesion recognition sequence
KRGWVWN

SEQ ID NO: 312 - protein, 8 aa, Artificial Sequence
  Atypical cadherin Trp-containing cell adhesion recognition sequence
KRGWVWNQ

SEQ ID NO: 313 - protein, 9 aa, Artificial Sequence
  Atypical cadherin Trp-containing cell adhesion recognition sequence
KRGWVWNQF

SEQ ID NO: 314 - protein, 10 aa, Artificial Sequence
  Atypical cadherin Trp-containing cell adhesion recognition sequence
KRGWVWNQFF

SEQ ID NO: 315 - protein, 11 aa, Artificial Sequence
  Atypical cadherin Trp-containing cell adhesion recognition sequence
KRGWVWNQFFV

SEQ ID NO: 316 - protein, 7 aa, Artificial Sequence
  Atypical cadherin Trp-containing cell adhesion recognition sequence
DWIWNQM

SEQ ID NO: 317 - protein, 8 aa, Artificial Sequence
  Atypical cadherin Trp-containing cell adhesion recognition sequence
DWIWNQMH

SEQ ID NO: 318 - protein, 9 aa, Artificial Sequence
  Atypical cadherin Trp-containing cell adhesion recognition sequence
DWIWNQMHI

SEQ ID NO: 319 - protein, 6 aa, Artificial Sequence
  Atypical cadherin Trp-containing cell adhesion recognition sequence
WIWNQM

SEQ ID NO: 320 - protein, 7 aa, Artificial Sequence
  Atypical cadherin Trp-containing cell adhesion recognition sequence
WIWNQMH

SEQ ID NO: 321 - protein, 8 aa, Artificial Sequence
  Atypical cadherin Trp-containing cell adhesion recognition sequence
WIWNQMHI

SEQ ID NO: 322 - protein, 4 aa, Artificial Sequence
  Atypical cadherin Trp-containing cell adhesion recognition sequence
RDWI

SEQ ID NO: 323 - protein, 5 aa, Artificial Sequence
  Atypical cadherin Trp-containing cell adhesion recognition sequence
RDWIW

SEQ ID NO: 324 - protein, 6 aa, Artificial Sequence
  Atypical cadherin Trp-containing cell adhesion recognition sequence
RDWIWN

SEQ ID NO: 325 - protein, 7 aa, Artificial Sequence
  Atypical cadherin Trp-containing cell adhesion recognition sequence
RDWIWNQ

SEQ ID NO: 326 - protein, 8 aa, Artificial Sequence
  Atypical cadherin Trp-containing cell adhesion recognition sequence
RDWIWNQM

SEQ ID NO: 327 - protein, 9 aa, Artificial Sequence
  Atypical cadherin Trp-containing cell adhesion recognition sequence
RDWIWNQMH

SEQ ID NO: 328 - protein, 10 aa, Artificial Sequence
  Atypical cadherin Trp-containing cell adhesion recognition sequence
RDWIWNQMHI

SEQ ID NO: 329 - protein, 4 aa, Artificial Sequence
  Atypical cadherin Trp-containing cell adhesion recognition sequence
KRDW

SEQ ID NO: 330 - protein, 5 aa, Artificial Sequence
  Atypical cadherin Trp-containing cell adhesion recognition sequence
KRDWI

SEQ ID NO: 331 - protein, 6 aa, Artificial Sequence
  Atypical cadherin Trp-containing cell adhesion recognition sequence
KRDWIW

SEQ ID NO: 332 - protein, 7 aa, Artificial Sequence
  Atypical cadherin Trp-containing cell adhesion recognition sequence
KRDWIWN

SEQ ID NO: 333 - protein, 8 aa, Artificial Sequence
  Atypical cadherin Trp-containing cell adhesion recognition sequence
KRDWIWNQ

SEQ ID NO: 334 - protein, 9 aa, Artificial Sequence
  Atypical cadherin Trp-containing cell adhesion recognition sequence
KRDWIWNQM

SEQ ID NO: 335 - protein, 10 aa, Artificial Sequence
  Atypical cadherin Trp-containing cell adhesion recognition sequence
KRDWIWNQMH

SEQ ID NO: 336 - protein, 11 aa, Artificial Sequence
  Atypical cadherin Trp-containing cell adhesion recognition sequence
KRDWIWNQMHI

SEQ ID NO: 337 - protein, 7 aa, Artificial Sequence
  Atypical cadherin Trp-containing cell adhesion recognition sequence
SWMWNQF

SEQ ID NO: 338 - protein, 8 aa, Artificial Sequence
  Atypical cadherin Trp-containing cell adhesion recognition sequence
SWMWNQFF

SEQ ID NO: 339 - protein, 9 aa, Artificial Sequence
  Atypical cadherin Trp-containing cell adhesion recognition sequence
SWMWNQFFL

SEQ ID NO: 340 - protein, 6 aa, Artificial Sequence
  Atypical cadherin Trp-containing cell adhesion recognition sequence
WMWNQF

SEQ ID NO: 341 - protein, 7 aa, Artificial Sequence
  Atypical cadherin Trp-containing cell adhesion recognition sequence
WMWNQFF

SEQ ID NO: 342 - protein, 8 aa, Artificial Sequence
  Atypical cadherin Trp-containing cell adhesion recognition sequence
WMWNQFFL

SEQ ID NO: 343 - protein, 4 aa, Artificial Sequence
  Atypical cadherin Trp-containing cell adhesion recognition sequence
RSWM

SEQ ID NO: 344 - protein, 5 aa, Artificial Sequence
  Atypical cadherin Trp-containing cell adhesion recognition sequence
RSWMW

SEQ ID NO: 345 - protein, 6 aa, Artificial Sequence
  Atypical cadherin Trp-containing cell adhesion recognition sequence
RSWMWN

SEQ ID NO: 346 - protein, 7 aa, Artificial Sequence
  Atypical cadherin Trp-containing cell adhesion recognition sequence
RSWMWNQ

SEQ ID NO: 347 - protein, 8 aa, Artificial Sequence
  Atypical cadherin Trp-containing cell adhesion recognition sequence
RSWMWNQF

SEQ ID NO: 348 - protein, 9 aa, Artificial Sequence
  Atypical cadherin Trp-containing cell adhesion recognition sequence
RSWMWNQFF

SEQ ID NO: 349 - protein, 10 aa, Artificial Sequence
  Atypical cadherin Trp-containing cell adhesion recognition sequence
RSWMWNQFFL

SEQ ID NO: 350 - protein, 4 aa, Artificial Sequence
  Atypical cadherin Trp-containing cell adhesion recognition sequence
KRSW

SEQ ID NO: 351 - protein, 5 aa, Artificial Sequence
  Atypical cadherin Trp-containing cell adhesion recognition sequence
KRSWM

SEQ ID NO: 352 - protein, 6 aa, Artificial Sequence
  Atypical cadherin Trp-containing cell adhesion recognition sequence
KRSWMW

SEQ ID NO: 353 - protein, 7 aa, Artificial Sequence
  Atypical cadherin Trp-containing cell adhesion recognition sequence
KRSWMWN

SEQ ID NO: 354 - protein, 8 aa, Artificial Sequence
  Atypical cadherin Trp-containing cell adhesion recognition sequence
KRSWMWNQ

SEQ ID NO: 355 - protein, 9 aa, Artificial Sequence
  Atypical cadherin Trp-containing cell adhesion recognition sequence
KRSWMWNQF

SEQ ID NO: 356 - protein, 10 aa, Artificial Sequence
  Atypical cadherin Trp-containing cell adhesion recognition sequence
KRSWMWNQFF

SEQ ID NO: 357 - protein, 11 aa, Artificial Sequence
  Atypical cadherin Trp-containing cell adhesion recognition sequence
KRSWMWNQFFL

SEQ ID NO: 358 - protein, 7 aa, Artificial Sequence
  Atypical cadherin Trp-containing cell adhesion recognition sequence
SWVWNQF

SEQ ID NO: 359 - protein, 8 aa, Artificial Sequence
  Atypical cadherin Trp-containing cell adhesion recognition sequence
SWVWNQFF

SEQ ID NO: 360 - protein, 9 aa, Artificial Sequence
  Atypical cadherin Trp-containing cell adhesion recognition sequence
SWVWNQFFV

SEQ ID NO: 361 - protein, 6 aa, Artificial Sequence
  Atypical cadherin Trp-containing cell adhesion recognition sequence
WVWNQF

SEQ ID NO: 362 - protein, 7 aa, Artificial Sequence
  Atypical cadherin Trp-containing cell adhesion recognition sequence
WVWNQFF

SEQ ID NO: 363 - protein, 8 aa, Artificial Sequence
  Atypical cadherin Trp-containing cell adhesion recognition sequence
WVWNQFFV

SEQ ID NO: 364 - protein, 4 aa, Artificial Sequence
  Atypical cadherin Trp-containing cell adhesion recognition sequence
RSWV

SEQ ID NO: 365 - protein, 5 aa, Artificial Sequence
  Atypical cadherin Trp-containing cell adhesion recognition sequence
RSWVW

SEQ ID NO: 366 - protein, 6 aa, Artificial Sequence
  Atypical cadherin Trp-containing cell adhesion recognition sequence
RSWVWN

SEQ ID NO: 367 - protein, 7 aa, Artificial Sequence
  Atypical cadherin Trp-containing cell adhesion recognition sequence
RSWVWNQ

SEQ ID NO: 368 - protein, 8 aa, Artificial Sequence
  Atypical cadherin Trp-containing cell adhesion recognition sequence
RSWVWNQF

SEQ ID NO: 369 - protein, 9 aa, Artificial Sequence
  Atypical cadherin Trp-containing cell adhesion recognition sequence
RSWVWNQFF

SEQ ID NO: 370 - protein, 10 aa, Artificial Sequence
  Atypical cadherin Trp-containing cell adhesion recognition sequence
RSWVWNQFFV

SEQ ID NO: 371 - protein, 5 aa, Artificial Sequence
  Atypical cadherin Trp-containing cell adhesion recognition sequence
KRSWV

SEQ ID NO: 372 - protein, 6 aa, Artificial Sequence
  Atypical cadherin Trp-containing cell adhesion recognition sequence
KRSWVW

SEQ ID NO: 373 - protein, 7 aa, Artificial Sequence
  Atypical cadherin Trp-containing cell adhesion recognition sequence
KRSWVWN

SEQ ID NO: 374 - protein, 8 aa, Artificial Sequence
  Atypical cadherin Trp-containing cell adhesion recognition sequence
KRSWVWNQ

SEQ ID NO: 375 - protein, 9 aa, Artificial Sequence
  Atypical cadherin Trp-containing cell adhesion recognition sequence
KRSWVWNQF

SEQ ID NO: 376 - protein, 10 aa, Artificial Sequence
  Atypical cadherin Trp-containing cell adhesion recognition sequence
KRSWVWNQFF

SEQ ID NO: 377 - protein, 11 aa, Artificial Sequence
  Atypical cadherin Trp-containing cell adhesion recognition sequence
KRSWVWNQFFV

SEQ ID NO: 378 - protein, 7 aa, Artificial Sequence
  Atypical cadherin Trp-containing cell adhesion recognition sequence
GWVWNQM

SEQ ID NO: 379 - protein, 8 aa, Artificial Sequence
  Atypical cadherin Trp-containing cell adhesion recognition sequence
GWVWNQMF

SEQ ID NO: 380 - protein, 9 aa, Artificial Sequence
  Atypical cadherin Trp-containing cell adhesion recognition sequence
GWVWNQMFV

SEQ ID NO: 381 - protein, 8 aa, Artificial Sequence
  Atypical cadherin Trp-containing cell adhesion recognition sequence
RGWVWNQM

SEQ ID NO: 382 - protein, 9 aa, Artificial Sequence
  Atypical cadherin Trp-containing cell adhesion recognition sequence
RGWVWNQMF

SEQ ID NO: 383 - protein, 10 aa, Artificial Sequence
  Atypical cadherin Trp-containing cell adhesion recognition sequence
RGWVWNQMFV

SEQ ID NO: 384 - protein, 9 aa, Artificial Sequence
  Atypical cadherin Trp-containing cell adhesion recognition sequence
KRGWVWNQM

SEQ ID NO: 385 - protein, 11 aa, Artificial Sequence
  Atypical cadherin Trp-containing cell adhesion recognition sequence
KRGWVWNQMFV

SEQ ID NO: 386 - protein, 9 aa, Artificial Sequence
  Atypical cadherin Trp-containing cell adhesion recognition sequence
GWVWNQFFL

SEQ ID NO: 387 - protein, 10 aa, Artificial Sequence
  Atypical cadherin Trp-containing cell adhesion recognition sequence
RGWVWNQFFL

SEQ ID NO: 388 - protein, 11 aa, Artificial Sequence
  Atypical cadherin Trp-containing cell adhesion recognition sequence
KRGWVWNQFFL

SEQ ID NO: 389 - protein, 7 aa, Artificial Sequence
  Atypical cadherin Trp-containing cell adhesion recognition sequence
AWVIPPI

SEQ ID NO: 390 - protein, 8 aa, Artificial Sequence
  Atypical cadherin Trp-containing cell adhesion recognition sequence
AWVIPPIS

SEQ ID NO: 391 - protein, 9 aa, Artificial Sequence
  Atypical cadherin Trp-containing cell adhesion recognition sequence
AWVIPPISV

SEQ ID NO: 392 - protein, 6 aa, Artificial Sequence
  Atypical cadherin Trp-containing cell adhesion recognition sequence
WVIPPI

SEQ ID NO: 393 - protein, 7 aa, Artificial Sequence
  Atypical cadherin Trp-containing cell adhesion recognition sequence
WVIPPIS

SEQ ID NO: 394 - protein, 8 aa, Artificial Sequence
  Atypical cadherin Trp-containing cell adhesion recognition sequence
WVIPPISV

SEQ ID NO: 395 - protein, 4 aa, Artificial Sequence
  Atypical cadherin Trp-containing cell adhesion recognition sequence
RAWV

SEQ ID NO: 396 - protein, 5 aa, Artificial Sequence
  Atypical cadherin Trp-containing cell adhesion recognition sequence
RAWVI

SEQ ID NO: 397 - protein, 6 aa, Artificial Sequence
  Atypical cadherin Trp-containing cell adhesion recognition sequence
RAWVIP

SEQ ID NO: 398 - protein, 7 aa, Artificial Sequence
  Atypical cadherin Trp-containing cell adhesion recognition sequence
RAWVIPP

SEQ ID NO: 399 - protein, 8 aa, Artificial Sequence
  Atypical cadherin Trp-containing cell adhesion recognition sequence
RAWVIPPI

SEQ ID NO: 400 - protein, 9 aa, Artificial Sequence
  Atypical cadherin Trp-containing cell adhesion recognition sequence
RAWVIPPIS

SEQ ID NO: 401 - protein, 10 aa, Artificial Sequence
  Atypical cadherin Trp-containing cell adhesion recognition sequence
RAWVIPPISV

SEQ ID NO: 402 - protein, 4 aa, Artificial Sequence
  Atypical cadherin Trp-containing cell adhesion recognition sequence
KRAW

SEQ ID NO: 403 - protein, 5 aa, Artificial Sequence
  Atypical cadherin Trp-containing cell adhesion recognition sequence
KRAWV

SEQ ID NO: 404 - protein, 6 aa, Artificial Sequence
  Atypical cadherin Trp-containing cell adhesion recognition sequence
KRAWVI

SEQ ID NO: 405 - protein, 7 aa, Artificial Sequence
  Atypical cadherin Trp-containing cell adhesion recognition sequence
KRAWVIP

SEQ ID NO: 406 - protein, 8 aa, Artificial Sequence
  Atypical cadherin Trp-containing cell adhesion recognition sequence
KRAWVIPP

SEQ ID NO: 407 - protein, 9 aa, Artificial Sequence
  Atypical cadherin Trp-containing cell adhesion recognition sequence
KRAWVIPPI

SEQ ID NO: 408 - protein, 10 aa, Artificial Sequence
  Atypical cadherin Trp-containing cell adhesion recognition sequence
KRAWVIPPIS

SEQ ID NO: 409 - protein, 4 aa, Artificial Sequence
  Atypical cadherin Trp-containing cell adhesion recognition sequence
VWNQ

SEQ ID NO: 410 - protein, 5 aa, Artificial Sequence
  Atypical cadherin Trp-containing cell adhesion recognition sequence
VWNQM

SEQ ID NO: 411 - protein, 5 aa, Artificial Sequence
  Atypical cadherin Trp-containing cell adhesion recognition sequence
VWNQF

SEQ ID NO: 412 - protein, 6 aa, Artificial Sequence
  Atypical cadherin Trp-containing cell adhesion recognition sequence
VWNQMF

SEQ ID NO: 413 - protein, 6 aa, Artificial Sequence
  Atypical cadherin Trp-containing cell adhesion recognition sequence
VWNQFF

SEQ ID NO: 414 - protein, 4 aa, Artificial Sequence
  Atypical cadherin Trp-containing cell adhesion recognition sequence
WNQM

SEQ ID NO: 415 - protein, 4 aa, Artificial Sequence
  Atypical cadherin Trp-containing cell adhesion recognition sequence
WNQF

SEQ ID NO: 416 - protein, 5 aa, Artificial Sequence
  Atypical cadherin Trp-containing cell adhesion recognition sequence
WNQFF

SEQ ID NO: 417 - protein, 4 aa, Artificial Sequence
  Atypical cadherin Trp-containing cell adhesion recognition sequence
IWNQ

SEQ ID NO: 418 - protein, 5 aa, Artificial Sequence
  Atypical cadherin Trp-containing cell adhesion recognition sequence
IWNQM

SEQ ID NO: 419 - protein, 6 aa, Artificial Sequence
  Atypical cadherin Trp-containing cell adhesion recognition sequence
IWNQMH

SEQ ID NO: 420 - protein, 4 aa, Artificial Sequence
  Atypical cadherin Trp-containing cell adhesion recognition sequence
WNQM

SEQ ID NO: 421 - protein, 5 aa, Artificial Sequence
  Atypical cadherin Trp-containing cell adhesion recognition sequence
WNQMH

SEQ ID NO: 422 - protein, 4 aa, Artificial Sequence
  Atypical cadherin Trp-containing cell adhesion recognition sequence
MWNQ

SEQ ID NO: 423 - protein, 5 aa, Artificial Sequence
  Atypical cadherin Trp-containing cell adhesion recognition sequence
MWNQF

SEQ ID NO: 424 - protein, 6 aa, Artificial Sequence
  Atypical cadherin Trp-containing cell adhesion recognition sequence
MWNQFF

(19) United States
(12) Patent Application Publication
(10) Pub. No.: US 2008/0194467 A1
(43) Pub. Date: Aug. 14, 2008

(54) CANCER TREATMENT METHODS USING CADHERIN ANTAGONISTS IN COMBINATION WITH ANTICANCER AGENTS

(75) Inventors: William Paul Peters, Fernandina Beach, FL (US); Brian Huber, Durham, NC (US); Douglas Scott Tyler, Hillsborough, NC (US)

Correspondence Address: SEED INTELLECTUAL PROPERTY LAW GROUP PLLC, 701 FIFTH AVE, SUITE 5400, SEATTLE, WA, 98104, US

(73) Assignees: ADHEREX TECHNOLOGIES, INC., Ottawa (CA); DEPARTMENT OF VETERANS AFFAIRS, Washington, DC (US)

(21) Appl. No.: 11/863,127

(22) Filed: Sep. 27, 2007

Related U.S. Application Data
(60) Provisional application No. 60/848,624, filed on Sep. 27, 2006.

(51) Int. Cl. A61K 38/12; A61K 31/197; A61P 35/00

(52) U.S. Cl. 514/9; 514/564; 514/2

(57) ABSTRACT

Improved methods for treating cancer which employ combinations comprising cadherin antagonists with certain anticancer agents or treatments are provided. The methods of the invention involve the administration of cadherin antagonist before, concurrent with, or after, administration of an anticancer agent or treatment and provide unexpectedly improved therapeutic benefit in the treatment of tumors growing in vivo.

### CROSS-REFERENCE TO RELATED APPLICATION

This application claims the benefit under 35 U.S.C. § 119(e) of U.S. Provisional Patent Application No. 60/848,624, filed Sep. 27, 2006, where this provisional application is incorporated herein by reference in its entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 100086_{—}425_SEQUENCE_LISTING.txt. The text file is 90 KB, was created on Sep. 27, 2007, and is being submitted electronically via EFS-Web, concurrent with the filing of the specification.

### BACKGROUND

1. Technical Field

The present invention relates generally to methods for treating cancer using cadherin antagonists in combination with anticancer agents or treatments.

2. Description of the Related Art

Cadherins are a superfamily of calcium-dependent cell adhesion molecules (CAMs) (for review, see Munro et al., In: Cell Adhesion and Invasion in Cancer Metastasis, P. Brodt, ed., pp. 17-34, RG Landes Co., Austin Tex., 1996; Rowlands T M. et al (2000) Rev. Reprod. 5: 53-61, Nollet F. et al (2000) J. Mol. Biol. 299: 551-572). All cadherins appear to be membrane glycoproteins that generally promote cell adhesion through homophilic interactions (a cadherin on the surface of one cell binds to an identical cadherin on the surface of another cell), although cadherins also appear to be capable of forming heterotypic complexes with one another under certain circumstances and with lower affinity.

There are many different types of cadherins. The most extensively studied group of cadherins is known as the classical, or type I, cadherins. Classical cadherins have been shown to regulate epithelial, endothelial, neural and cancer cell adhesion, with different cadherins expressed on different cell types. All classical cadherins have a similar structure. Classical cadherins are composed of five extracellular domains (EC1-EC5), a single hydrophobic domain (TM) that transverses the plasma membrane (PM), and two cytoplasmic domains (CP1 and CP2). Calcium binding motifs are interspersed throughout the extracellular domains, and each 110 amino acid region that contains such motifs is considered a cadherin repeat. The first extracellular domain (EC1) contains the cell adhesion recognition (CAR) sequence, HAV (His-Ala-Val), along with flanking sequences on either side of the CAR sequence that play a role in conferring specificity. Synthetic peptides containing the HAV sequence and antibodies directed against such peptides have been shown to inhibit classical cadherin-dependent processes (Munro et al., supra; Blaschuk et al., J. Mol. Biol. 211:679-82, 1990; Blaschuk et al., Develop. Biol. 139:227-29, 1990; Alexander et al., J. Cell. Physiol. 156:610-18, 1993; Makrigiannakis. et al. (1999) Am. J. Pathol. 154: 1391-1406; Wilby et al. (1999) Mol. Cell. Neurosci. 14: 66-84; Schnädelbach et al (2000) Mol. Cell. Neurosci. 15: 288-302; Williams et al. (2000) J. Biol. Chem. 275: 4007-4012; Schnädelbach et al. (2001) Mol. Cell. Neurosci. 17: 1084-1093; Erez et al. Exp. Cell Res. 294: 366-78; see also U.S. Pat. Nos. 6,031,072; 6,169,071; 6,417,325).

Cadherins that contain calcium binding motifs within extracellular domain cadherin repeats, but do not contain an HAV CAR sequence, are considered to be nonclassical cadherins. At least six groups of nonclassical cadherins have been identified as well several other cadherins that are not classified within the six groups These cadherins are also membrane glycoproteins. Type II, or atypical, cadherins include OB-cadherin (cadherin-11; see Getsios et al., Developmental Dynamics 211:238-247, 1998; Simonneau et al., Cell Adhesion and Communication 3:115-130, 1995; Okazaki et al., J. Biological Chemistry 269:12092-12098, 1994), cadherin-5 (VE-cadherin; see Navarro et al., J. Cell Biology 140:1475-1484, 1998), cadherin-6 (K-cadherin; see Shimoyama et al., Cancer Research 55:2206-2211, 1995; Shimazui et al., Cancer Research 56:3234-3237, 1996; Inoue et al., Developmental Dynamics 211:338-351, 1998; Getsios et al., Developmental Dynamics 211:238-247, 1998), cadherin-7 (see Nakagawa et al., Development 121:1321-1332, 1995), cadherin-8 (see Suzuki et al., Cell Regulation 2:261-270, 1991), cadherin-12 (Br-cadherin; see Tanihara et al., Cell Adhesion and Communication 2:15-26, 1994), cadherin-14 (see Shibata et al., J. Biological Chemistry 272:5236-5240, 1997), cadherin-15 (M-cadherin; see Shimoyama et al., J. Biological Chemistry 273:10011-10018, 1998), and PB-cadherin (see Sugimoto et al., J. Biological Chemistry 271:11548-11556, 1996). For a general review of atypical cadherins, see Redies and Takeichi, Developmental Biology 180:413-423, 1996; Suzuki et al., Cell Regulation 2:261-270, 1991; Nollet F. et al, (2000) J. Mol. Biol. 299: 551-572.

OB-cadherin, which is also known as cadherin-11, is an atypical cadherin (Getsios et al., Developmental Dynamics 211:238-247, 1998; Okazaki et al., J. Biol. Chem. 269:12092-98, 1994; Suzuki et al., Cell Regulation 2:261-70, 1991; Munro et al., supra). This cadherin can promote cell adhesion through homophilic interactions. OB-cadherin does not contain the classical cadherin cell adhesion recognition sequence, HAV. A unique feature of OB-cadherin is the existence of two alternatively spliced isoforms: a full-length form with a cytoplasmic domain that interacts with catenins; and a truncated form that lacks most of the cytoplasmic domain (Feltes et al., Cancer Research 62:6688-6697, 2002). The truncated OB-cadherin variant is also shed from the cell surface and can be found deposited in the extracellular matrix surrounding the cells.

Vascular endothelial cadherin (VE-cadherin also known as cadherin-5) is an endothelial specific cadherin localized at intracellular junctions of essentially all types of endothelium, including the endothelium of blood vessels and of lymphatic vessels. VE-cadherin has been shown to be localized at certain intercellular junctions-adherens junctions (AJ) in cell-to-cell contacts. A number of observations suggest that VE-cadherin is involved in various aspects of vascular biology related to endothelial cell adhesion, angiogenesis, maintenance of vascular integrity and regulation of vascular permeability. In addition to mediating inter-endothelial homotypic cell-cell adhesion, VE-cadherin interacts with and influences the activity of growth factor receptors on the surface of endothelial cells. For instance, VE-cadherin is required for intracellular signals from vascular endothelial growth factor (VEGF) via vascular endothelial growth factor receptor-2 (VEGF-R2) leading to survival of endothelial cells (Carmeliet et al. Cell. 1999 Jul. 23; 98(2):147-57) and VE-cadherin may influence signals from growth factors that regulate the migration and proliferation of endothelial cells (Zanetti et al. Arterioscler Thromb Vasc Biol. 2002 Apr. 1; 22(4):617-22). VEGF family members and their receptors are central signalers in the angiogenic process (Carmeliet and Jain, Nature. 2000 Sep. 14; 407(6801):249-57) Collectively, these and other observations underscore the importance of VE-cadherin as a target for the development of novel agents for treating human diseases such as cancer, psoriasis, age-related macular degeneration, ischaemic heart disease, ischaemic limb disease, warts, ulcers, endometriosis, follicular cysts, adhesions, uterine bleeding, atherosclerosis, keloids, ovarian hyperstimulation, peritoneal sclerosis, arthritis, asthma, retinopathy, stroke, lymphoproliferative disorders, lymphodema, thyroid enlargement, intraocular disorders, pulmonary hypertension, healing of bone fractures and obesity.

Cancer is a significant health problem throughout the world. Management of the disease currently relies on a combination of early diagnosis (through various screening procedures) and aggressive treatment, which may include one or more of a variety of treatments such as surgery, radiotherapy, chemotherapy and/or hormone therapy. Numerous therapeutic agents and strategies have been used in treating and managing human cancers, however no universally successful methods have been identified and improved approaches are continually being sought. Therapies involving specific combinations of distinct anticancer agents have proven highly effective in certain instances, however, identification of the specific combinations that provide synergistic advantages has been largely unpredictable.

Thus, despite numerous advances in the identification and commercialization of successful cancer therapeutic agents, there remains a significant and unmet need for identifying combinations of therapeutic agents and/or other treatment modalities that provide benefits that are greater than would be expected based upon the performance of the agents or treatments individually. There also is a significant need in the art to identify combinations of agents that are effective for minimizing or overcoming natural and acquired resistance to conventional chemotherapies.

The present invention fulfills these needs and further provides other related advantages.

### BRIEF SUMMARY

The present invention is drawn generally to combination therapies for the treatment of human cancers, said therapies comprising the administration of cadherin antagonists in conjunction other anticancer agents or treatments. The therapeutic benefits observed according to the methods of the invention are improved to an unexpected extent relative to the use of the agents or treatments individually. The present invention thus provides valuable new therapeutic strategies for managing cancer. Therefore, according to one aspect of the invention, there is provided a method for the treatment of a cancer comprising administering to a subject in need thereof at least one cadherin antagonist and at least one anticancer alkylating agent. The alkylating agent may be selected, for example, from agents such as mechlorethamine, cyclophosphamide, ifosfamide, trofosfamide, melphalan (L-sarcolysin), chlorambucil, hexamethylmelamine, thiotepa, busulfan, carmustine (BCNU), streptozocin (streptozotocin), dacarbazine (DTIC; dimethyltriazenoimid-azolecarboxamide) and temozolomide. In one preferred embodiment, the agent is melphalan.

According to another aspect of the invention, there is provided a method for the treatment of a cancer comprising administering to a subject in need thereof at least one cadherin antagonist and at least one anticancer antimetabolite, such as an agent selected from pyrimidine analogs and purine analogs. In a particular embodiment, the anticancer antimetabolite is selected from the group consisting of fluorouracil, 5-fluorouracil, floxuridine (fluoride-oxyuridine; FUdR), capecitabine, pemetrexed, cytarabine (cytosine arabinoside), gemcitabine, mercaptopurine (6-mercaptopurine; 6-MP) and thioguanine.

According to another aspect of the invention, there is provided a method for the treatment of a cancer comprising administering to a subject in need thereof at least one cadherin antagonist and at least one anticancer natural product, such as an agent selected from vinca alkaloids, taxanes, epipodophylltoxins, camptothecins antibiotics, enzymes, biological response modifiers and immunostimulators. In one embodiment, the anticancer natural product is selected from the group consisting of docataxel, etoposide, teniposide; topotecan, irinotecan, dactinomycin (actinomycin D), daunorubicin (daunomycin; rubidomycin), doxorubicin, bleomycin, L-Asparaginase, interferon-alfa and interleukin 2. In a particular embodiment, the natural product anticancer agent is not taxol or a vinca alkaloid.

In another aspect, the invention provides a method for the treatment of a cancer comprising administering to a subject in need thereof at least one cadherin antagonist and at least one anticancer antibiotic, such as an agent selected from eiprubicin, idarubicin and liposomal doxorubicin.

In another aspect, the invention provides a method for the treatment of a cancer comprising administering to a subject in need thereof at least one cadherin antagonist and at least one agent selected from the group consisting of plantinum compounds, anthracenediones, methylhydrazine derivatives, adrenocortical suppressants, tyrosine kinase inhibitors, multi-targeted kinase inhibitors, adrenocorticosteroids, estrogens, progestins, aromatase inhibitors, antiestrogens, antitumor antibodies and radiation therapy. In one embodiment, the platinum compound is selected from the group consisting of cisplatin (cis-DDP), carboplatin and oxaliplatin.

In a more particular embodiment, the anthracenedione is mitoxantrone. In another embodiment, the methylhydrazine derivative is N-methylhydrazine (MIH). In yet another embodiment, the adrenocortical suppressant is selected from the group consisting of mitotane and aminoglutethimide. In another embodiment, the tyrosine kinase inhibitor is selected from the group consisting of imatinib, erlotinib and gefitinib. In another embodiment, the multi-targeted kinase inhibitor is selected from the group consisting of sunitinib, sorafanib and dasatinib. In another embodiment, the adrenocorticosteriods is selected from the group consisting of prednisone and prednisolone. In another embodiment, the estrogen is diethylstilbestrol. In another embodiment, the progestin is megestrol acetate. In another embodiment, the aromatase inhibitor is selected from the group consisting of exemestane and letrozole. In another embodiment, the antiestrogen is tamoxifen. In another embodiment, the anticancer antibody is selected from the group consisting of bevacizumab, rituximab, cetuximab, panitumomab and ¹³¹I-tositumomab.

In another aspect, the invention provides a method for the treatment of a cancer comprising administering to a subject in need thereof at least one cadherin antagonist in combination with radiation therapy.

The cadherin antagonist employed in the methods of the invention, in certain embodiments, is a peptide comprising the sequence HAV, such as a cyclic peptide comprising the sequence HAV. Illustratively, the antagonist may be a cyclic peptide having the formula:

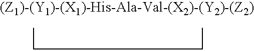

wherein X₁, and X₂ are optional, and if present, are independently selected from the group consisting of amino acid residues and combinations thereof in which the residues are linked by peptide bonds, and wherein X₁ and X₂ independently range in size from 0 to 10 residues, such that the sum of residues contained within X₁ and X₂ ranges from 1 to 12; wherein Y₁ and Y₂ are independently selected from the group consisting of amino acid residues, and wherein a covalent bond is formed between residues Y₁ and Y₂; and wherein Z₁ and Z₂ are optional, and if present, are independently selected from the group consisting of amino acid residues and combinations thereof in which the residues are linked by peptide bonds.

Other illustrative cyclic peptides comprise a sequence selected from the group consisting of: N—Ac-CHAVC-NH₂ (SEQ ID NO:1). Another preferred cyclic peptide is N—Ac-CHAVC-Y—NH₂ (SEQ ID NO:2). Other cyclic peptides include, but are not limited to: N—Ac-CHAVDC-NH₂ (SEQ ID NO:3), N—Ac-CHAVDIC-NH₂ (SEQ ID NO:4), N—Ac-CHAVDINC-NH₂ (SEQ ID NO:5), N—Ac-CHAVDINGC-NH₂ (SEQ ID NO:6), N—Ac-CAHAVC-NH₂ (SEQ ID NO:7), N—Ac-CAHAVDC-NH₂ (SEQ ID NO:8), N—Ac-CAHAVDIC-NH₂ (SEQ ID NO:9), N—Ac-CRAHAVDC-NH₂ (SEQ ID NO:10), N—Ac-CLRAHAVC-NH₂ (SEQ ID NO:11), N—Ac-CLRAHAVDC-NH₂ (SEQ ID NO:12), N—Ac-CSHAVC-NH₂ (SEQ ID NO:13), N—Ac-CFSHAVC-NH₂ (SEQ ID NO:14), N—Ac-CLFSHAVC-NH₂ (SEQ ID NO:15), N—Ac-CHAVSC-NH₂ (SEQ ID NO:16), N—Ac-CSHAVSC-NH₂ (SEQ ID NO:17), N—Ac-CSHAVSSC-NH₂ (SEQ ID NO:18), N—Ac-CHAVSSC-NH₂ (SEQ ID NO:19), N—Ac-KHAVD-NH₂ (SEQ ID NO:20), N—Ac-DHAVK-NH₂ (SEQ ID NO:21), N—Ac-KHAVE-NH₂ (SEQ ID NO:22), N—Ac-AHAVDI-NH₂ (SEQ ID NO:23), N—Ac-SHAVDSS-NH₂ (SEQ ID NO:24), N—Ac-KSHAVSSD-NH₂ (SEQ ID NO:25), N—Ac-CHAVC-S—NH₂ (SEQ ID NO:26), N—Ac—S-CHAVC-NH₂ (SEQ ID NO:27), N—Ac-CHAVC-SS—NH₂ (SEQ ID NO:28), N—Ac—S-CHAVC-S—NH₂ (SEQ ID NO:29), N—Ac-CHAVC-T-NH₂ (SEQ ID NO:30), N—Ac-CHAVC-E-NH₂ (SEQ ID NO:31), N—Ac-CHAVC-D-NH₂ (SEQ ID NO:32), N—Ac-CHAVYC-NH₂ (SEQ ID NO:33), CH₃—SO₂—HN—CHAVC-Y—NH₂ (SEQ ID NO:34), CH₃—SO₂—HN-CHAVC-NH₂ (SEQ ID NO:35), HC(O)—NH-CHAVC-NH₂ (SEQ ID NO:36), N—Ac-CHAVPen-NH₂ (SEQ ID NO:37), N—Ac-PenHAVC-NH₂ (SEQ ID NO:38) and N—Ac-CHAVPC-NH₂ (SEQ ID NO:39). In one preferred embodiment, the cyclic peptide comprises the sequence N—Ac-CHAVC-NH₂ (SEQ ID NO:1).

Other cadherin antagonists useful in the methods of the invention include antagonists comprising the sequence Asp/Glu-Trp-Val-Ile/Val/Met-Pro/Ala-Pro (SEQ ID NO:40), wherein “Asp/Glu” is an amino acid that is either Asp or Glu, “Ile/Val/Met” is an amino acid that is Ile, Val or Met, and “Pro/Ala” is either Pro or Ala. In one illustrative embodiment, the cadherin antagonist comprises a sequence selected from the group consisting of DWV, DWVI (SEQ ID NO:41), DWVV (SEQ ID NO: _{—}42), DWVM (SEQ ID NO:43), DWVIP (SEQ ID NO:44), DWVIA (SEQ ID NO:45), DWVVP (SEQ ID NO:46), DWVVPP (SEQ ID NO:47), DWVVAP (SEQ ID NO:48), DWVMPP (SEQ ID NO:49), DWVMAP (SEQ ID NO:50), EWV, EWVI (SEQ ID NO:51), EWVV (SEQ ID NO:52), EWVM (SEQ ID NO:53), EWVIP (SEQ ID NO:54), EWVIA (SEQ ID NO:55), EWVVP (SEQ ID NO:56), EWVVPP (SEQ ID NO:57), EWVVAP (SEQ ID NO:58), EWVMPP (SEQ ID NO:59), EWVMAP (SEQ ID NO:60), WVI, WVIP (SEQ ID NO:61), WVIA (SEQ ID NO:62), WVV, WVVP (SEQ ID NO:63), WVVA (SEQ ID NO:64), WVM, WVMP (SEQ ID NO:65), WVMA (SEQ ID NO:66), WVIPP (SEQ ID NO:67), WVIAP (SEQ ID NO:68), WVVPP (SEQ ID NO:69), WVVAP (SEQ ID NO:70), WVMPP (SEQ ID NO:71), WVMAP (SEQ ID NO:72), DWI, DWII (SEQ ID NO:73), DWIV (SEQ ID NO:74), DWIM (SEQ ID NO:75), DWIIP (SEQ ID NO:76), DWIIA (SEQ ID NO:77), DWIVP (SEQ ID NO:78), DWIVPP (SEQ ID NO:79), DWIVAP (SEQ ID NO:80), DWIMPP (SEQ ID NO:81), DWIMAP (SEQ ID NO:82), EWI, EWII (SEQ ID NO:83), EWIV (SEQ ID NO:84), EWIM (SEQ ID NO:85), EWIIP (SEQ ID NO:86), EWIIA (SEQ ID NO:87), EWIVP (SEQ ID NO:88), EWIVPP (SEQ ID NO:89), EWIVAP (SEQ ID NO:90), EWIMPP (SEQ ID NO:91), EWIMAP (SEQ ID NO:92), WII, WIIP (SEQ ID NO:93), WIIA (SEQ ID NO:94), WIV, WIVP (SEQ ID NO:95), WIVA (SEQ ID NO:96), WIM, WIMP (SEQ ID NO:97), WIMA (SEQ ID NO:98), WIIPP (SEQ ID NO:99), WIIAP (SEQ ID NO:100), WIVPP (SEQ ID NO:101), WIVAP (SEQ ID NO:102), WIMPP (SEQ ID NO:103), WIMAP (SEQ ID NO:104), DWL, DWLI (SEQ ID NO:105), DWLV (SEQ ID NO:106), DWLM (SEQ ID NO:107), DWLIP (SEQ ID NO:108), DWLIA (SEQ ID NO:109), DWLVP (SEQ ID NO: 110), DWLVPP (SEQ ID NO:111), DWLVAP (SEQ ID NO:112), DWLMPP (SEQ ID NO:113), DWLMAP (SEQ ID NO:114), EWL, EWLI (SEQ ID NO:115), EWLV (SEQ ID NO:116), EWLM (SEQ ID NO:117), EWLIP (SEQ ID NO:118), EWLIA (SEQ ID NO:119), EWLVP (SEQ ID NO:120), EWLVPP (SEQ ID NO:121), EWLVAP (SEQ ID NO:122), EWLMPP (SEQ ID NO:123), EWLMAP (SEQ ID NO:124), WLI, WLIP (SEQ ID NO:125), WLIA (SEQ ID NO:126), WLV, WLVP (SEQ ID NO:127), WLVA (SEQ ID NO:128), WLM, WLMP (SEQ ID NO:129), WLMA (SEQ ID NO:130), WLIPP (SEQ ID NO:131), WLIAP (SEQ ID NO:132), WLVPP (SEQ ID NO:133), WLVAP (SEQ ID NO:134), WLMPP (SEQ ID NO:135), WLMAP (SEQ ID NO:136), DWVL (SEQ ID NO:137), DWIL (SEQ ID NO:138), DWLL (SEQ ID NO:139), EWVL (SEQ ID NO:140), EWIL (SEQ ID NO:141), EWLL (SEQ ID NO:142), DWVLP (SEQ ID NO:143), DWILP (SEQ ID NO:144), DWLLP (SEQ ID NO:145), EWVLP (SEQ ID NO:146), EWILP (SEQ ID NO:147), EWLLP (SEQ ID NO:148), DWVLA (SEQ ID NO:149), DWILA (SEQ ID NO:150), DWLLA (SEQ ID NO:151), EWVLA (SEQ ID NO:152), EWILA (SEQ ID NO:153), EWLLA (SEQ ID NO:154), DWVLPP (SEQ ID NO:155), DWILPP (SEQ ID NO:156), DWLLPP (SEQ ID NO:157), EWVLPP (SEQ ID NO:158), EWILPP (SEQ ID NO:159), EWLLPP (SEQ ID NO:160), DWVLAP (SEQ ID NO:161), DWILAP (SEQ ID NO:162), DWLLAP (SEQ ID NO:163), EWVLAP (SEQ ID NO:164), EWILAP (SEQ ID NO:165), EWLLAP (SEQ ID NO:166), WVL, WIL, WLL, WVLP (SEQ ID NO:167), WILP (SEQ ID NO:168), WLLP (SEQ ID NO:169), WVLA (SEQ ID NO:170), WILA (SEQ ID NO:171), WLLA (SEQ ID NO:172), WVLPP (SEQ ID NO:173), WILPP (SEQ ID NO:174), WLLPP (SEQ ID NO:175), WVLAP (SEQ ID NO:176), WILAP (SEQ ID NO:177), and WLLAP (SEQ ID NO:178).

Other illustrative cadherin antagonist comprise a cyclic peptide having a structure selected from the group consisting of:

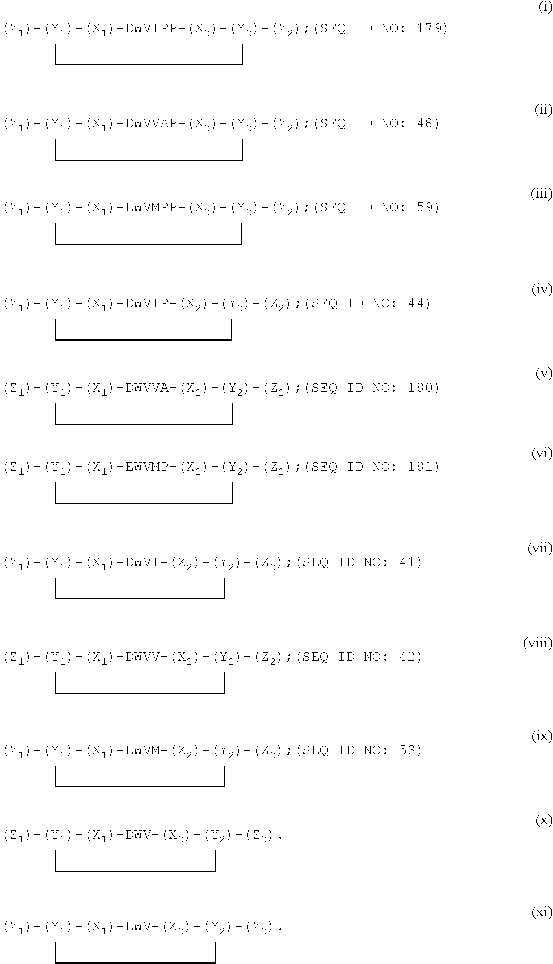

wherein X₁ and X₂ are optional, and if present, are amino acid residues, wherein X₁ and X₂ independently range in size from 0 to 10 residues, such that the sum of residues contained within X₁ and X₂ ranges from 1 to 12; Y₁ and Y₂ are amino acid residues, and a covalent bond is formed between residues Y₁ and Y₂; and Z₁ and Z₂ are optional, and if present, are amino acid residues linked by peptide bonds.

Still other illustrative cadherin antagonist comprise an HAV-BM sequence selected from the group consisting of: (a) Ile/Val-Phe-Aaa-Ile-Baa-Caa-Daa-Ser/Thr-Gly-Eaa-Leu/Met (SEQ ID NO:182), wherein Aaa, Baa, Caa, Daa and Eaa are independently selected from the group consisting of amino acid residues; or (b) Trp-Leu-Aaa-Ile-Asp/Asn-Baa-Caa-Daa-Gly-Gln-Ile (SEQ ID NO:183), wherein Aaa, Baa, Caa and Daa are independently selected from the group consisting of amino acid residues. In a particular embodiment, the cadherin antagonist comprises an HAV-BM sequence selected from the group consisting of: IFIINPISGQL (SEQ ID NO:184), IFILNPISGQL (SEQ ID NO:185), VFAVEKETGWL (SEQ ID NO:186), VFSINSMSGRM (SEQ ID NO:187), VFIIERETGWL (SEQ ID NO:188), VFTIEKESGWL (SEQ ID NO:189), VFNIDSMSGRM (SEQ ID NO:190), WLKIDSVNGQI (SEQ ID NO:191), WLKIDPVNGQI (SEQ ID NO:192), WLAMDPDSGQV (SEQ ID NO:193), WLHINATNGQI (SEQ ID NO:194), WLEINPDTGAI (SEQ ID NO:195), WLAVDPDSGQI (SEQ ID NO:196), WLEINPETGAI (SEQ ID NO:197), WLHINTSNGQI (SEQ ID NO:198), NLKIDPVNGQI (SEQ ID NO:199), LKIDPVNGQI (SEQ ID NO:200) INPISGQ (SEQ ID NO:201), LNPISGQ (SEQ ID NO:202), IDPVSGQ (SEQ ID NO:203) and KIDPVNGQ (SEQ ID NO:204), PISGQ (SEQ ID NO:205), PVNGQ (SEQ ID NO:206), PVSGR (SEQ ID NO:207), IDPVN (SEQ ID NO:208), INPIS (SEQ ID NO:209) and KIDPV (SEQ ID NO:210).

Still other illustrative cadherin antagonists comprise a cyclic peptide having a structure selected from the group consisting of:

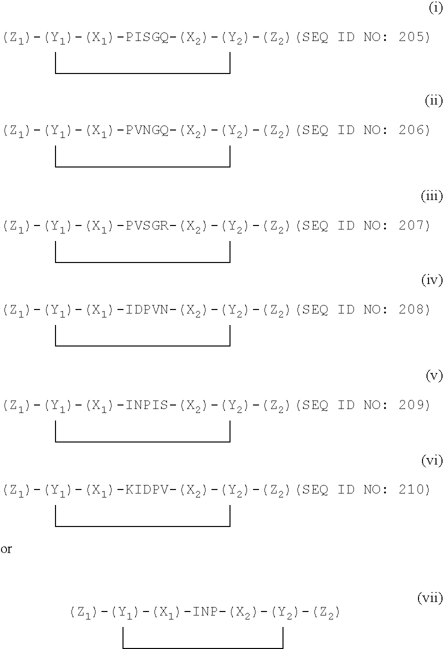

wherein X₁, and X₂ are optional, and if present, are independently selected from the group consisting of amino acid residues and combinations thereof in which the residues are linked by peptide bonds, wherein X₁ and X₂ independently range in size from 0 to 10 residues, such that the sum of residues contained within X₁ and X₂ ranges from 1 to 12; wherein Y₁ and Y₂ are independently selected from the group consisting of amino acid residues, and wherein a covalent bond is formed between residues Y₁ and Y₂; and wherein Z₁ and Z₂ are optional, and if present, are independently selected from the group consisting of amino acid residues and combinations thereof in which the residues are linked by peptide bonds.

Other illustrative cadherin antagonists comprise the sequence:

Aaa-Phe-Baa-Ile/Leu/Val-Asp/Asn/Glu-Caa-Daa-Ser/Thr/Asn-Gly (SEQ ID NO:211)

wherein Aaa, Baa, Caa and Daa are independently selected amino acid residues; Ile/Leu/Val is an amino acid that is selected from the group consisting of isoleucine, leucine and valine, Asp/Asn/Glu is an amino acid that is selected from the group consisting of aspartate, asparagine and glutamate; and Ser/Thr/Asn is an amino acid that is selected from the group consisting of serine, threonine or asparagine.

Still other illustrative cadherin antagonists have the formula:

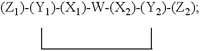

wherein W is a tripeptide selected from the group consisting of EEY, DDK, EAQ, DAE, NEN, ESE, DSG, DEN, EPK, DAN, EEF, NDV, DET, DPK, DDT, DAN, DKF, DEL, DAD, NNK, DLV, NRD, DPS, NQK, NRN, NKD, EKD, ERD, DPV, DSV, DLY, DSN, DSS, DEK, NEK; RAL, YAL, YAT, FAT and YAS wherein X₁, and X₂ are optional, and if present, are independently selected from the group consisting of amino acid residues and combinations thereof in which the residues are linked by peptide bonds, and wherein X₁ and X₂ independently range in size from 0 to 10 residues, such that the sum of residues contained within X₁ and X₂ ranges from 1 to 12; wherein Y₁ and Y₂ are independently selected from the group consisting of amino acid residues, and wherein a covalent bond is formed between residues Y₁ and Y₂; and wherein Z₁ and Z₂ are optional, and if present, are independently selected from the group consisting of amino acid residues and combinations thereof in which the residues are linked by peptide bonds.

In a more particular embodiment, the cadherin antagonist comprises a sequence selected from the group consisting of: DDK, IDDK (SEQ ID NO:212) DDKS (SEQ ID NO:213), VIDDK (SEQ ID NO:214), IDDKS (SEQ ID NO:215), VIDDKS (SEQ ID NO:216), DDKSG (SEQ ID NO:217), IDDKSG (SEQ ID NO:218), VIDDKSG (SEQ ID NO:219), FVIDDK (SEQ ID NO:220), FVIDDKS (SEQ ID NO:221), FVIDDKSG (SEQ ID NO:222), IFVIDDK (SEQ ID NO:223), IFVIDDKS (SEQ ID NO:224), IFVIDDKSG (SEQ ID NO:225), EEY, IEEY (SEQ ID NO:226), EEYT (SEQ ID NO:227), VIEEY (SEQ ID NO:228), IEEYT (SEQ ID NO:229), VIEEYT (SEQ ID NO:230), EEYTG (SEQ ID NO:231), IEEYTG (SEQ ID NO:232), VIEEYTG (SEQ ID NO:233), FVIEEY (SEQ ID NO:234), FVIEEYT (SEQ ID NO:235), FVIEEYTG (SEQ ID NO:236), FFVIEEY (SEQ ID NO:237), FFVIEEYT (SEQ ID NO:238), FFVIEEYTG (SEQ ID NO:239), EAQ, VEAQ (SEQ ID NO:240), EAQT (SEQ ID NO:241), SVEAQ (SEQ ID NO:242), VEAQT (SEQ ID NO:243), SVEAQT (SEQ ID NO:244), EAQTG (SEQ ID NO:245), VEAQTG (SEQ ID NO:246), SVEAQTG (SEQ ID NO:247), FSVEAQ (SEQ ID NO:248), FSVEAQT (SEQ ID NO:249), FSVEAQTG (SEQ ID NO:250), YFSVEAQ (SEQ ID NO:251), YFSVEAQT (SEQ ID NO:252) and YFSVEAQTG (SEQ ID NO:253).

In other embodiments, the cadherin antagonist comprises a sequence selected from the group consisting of DAE, VDAE (SEQ ID NO:254), DAET (SEQ ID NO:255), RVDAE (SEQ ID NO:256), VDAET (SEQ ID NO:257), RVDAET (SEQ ID NO:258), DAETG (SEQ ID NO:259), VDAETG (SEQ ID NO:260), RVDAETG (SEQ ID NO:261), FRVDAE (SEQ ID NO:262), FRVDAET (SEQ ID NO:263), FRVDAETG (SEQ ID NO:264), VFRVDAE (SEQ ID NO:265), VFRVDAET (SEQ ID NO:266) and VFRVDAETG (SEQ ID NO:267).

The methods of the invention may be employed in the treatment of a primary tumor or a metastatic cancer. In addition, the methods may be employed in treating chemoresistant tumors.

The cadherin antagonists are used in pharmaceutically effective amounts in the methods of the invention. Illustratively, the cadherin antagonist is administered at a dose between about 10-2500 mg/m².

The cadherin antagonist may be administered prior to administration of anticancer agent, for example about 7 days to about 1 hour prior to administration of anticancer agent. Alternatively, the cadherin antagonist may be administered after administration of anticancer agent, for example about 1 hour to about 3 weeks after administration of anticancer agent. In another embodiment, the cadherin antagonist is administered within about 1 hour of administration of anticancer agent.

The cancer to be treated according to the methods of the invention may be essentially any cancer type for which the combinations described herein offer desired and/or synergistic efficacy, including breast cancer, prostate cancer, skin cancer (e.g., basal cell carcinoma or melanoma), lung cancer (e.g., small cell or non-small cell), pancreatic cancer, kidney cancer, CNS cancer (e.g., glioma, neuroblastoma or astrocytoma), hepatocellular cancer, adrenocortical cancer, gastric cancer, esophageal cancer or ovarian cancer.

In another aspect of the invention, there are provide pharmaceutical formulations comprising at least one cadherin antagonist, as described herein, and at least one anticancer agent, as described herein. For example, illustrative formulations may comprise a cadherin antagonist and an anticancer agent selected from the group consisting of an alkylating agent, an anticancer antimetabolite, an anticancer natural product, an anticancer antibiotic, plantinum compounds, anthracenediones, methylhydrazine derivatives, adrenocortical suppressants, tyrosine kinase inhibitors, multi-targeted kinase inhibitors, adrenocorticosteroids, estrogens, progestins, aromatase inhibitors, antiestrogens and antitumor antibodies.

These and other aspects of the invention will become evident upon reference to the following detailed description and attached drawings. All references disclosed herein are hereby incorporated by reference in their entirety as if each were individually noted for incorporation.

### BRIEF DESCRIPTION OF THE SEVERAL VIEWS OF THE DRAWINGS

FIG. 1 shows illustrative design and dosing information of an isolated limb reperfusion treatment procedure using melphalan (LPAM) in combination with ADH-1 (N—Ac-CHAVC-NH₂); SEQ ID NO: 1).

FIG. 2A-2B show in vivo tumor growth curves for animals treated using melphalan alone or in combination with ADH-1 infused into the hind limb.

FIGS. 3A-3C show in vivo tumor growth curves for animals bearing tumors derived from the melanoma cell line DM738. The animals were treated using melphalan alone, ADH-1 alone or melphalan in combination with ADH-1.

FIG. 4 shows a Kaplan Meyer survival curve and illustrates that although A375 tumors are resistant to both temozolomide (TMZ) and ADH-1 when administered separately, survival is significantly improved when the agents are used in combination.

FIG. 5A-5B show in vivo tumor growth curves for animals treated using paclitaxel alone or in combination with ADH-1.

### DETAILED DESCRIPTION

As noted above, the present invention provides improved methods for treating cancer, and for overcoming or reducing chemoresistance, which employ combinations comprising cadherin antagonists, such as N-cadherin, VE-cadherin and/or OB-cadherin antagonists, with certain conventional anticancer agents or treatments. The methods are particularly advantageous in improving the efficacy of cancer treatment without increasing toxicity. As further described below, the methods of the invention involve the administration of a cadherin antagonist before, concurrent with, or after, administration of an anticancer agent or treatment. The use of a cadherin antagonist in conjunction with particular anticancer agents according to the invention provide unexpectedly improved therapeutic benefit in the treatment of tumors growing in vivo.

### 1. Cadherin Antagonists

A cadherin antagonist used according to the invention may include essentially any compound capable of modulating a cadherin protein, particularly compounds capable of inhibiting at least one cadherin-mediated function or process, such as cell adhesion. Illustrative examples of various known cadherin antagonists that may be used in the present invention are described below.

a. Cadherin Antagonists Comprising HAV CAR Sequences

Certain peptide-based cadherin antagonists have been extensively described and are useful in the present invention, e.g., U.S. Pat. Nos. 6,031,072; 6,417,325; 6,465,427; 6,780,845; 6,203,788; and WO05/012348, the contents of which are incorporated herein by reference in their entireties. Such agents represent classical cadherin antagonists and generally comprise linear and/or cyclic peptides containing the classical cadherin cell adhesion recognition (CAR) sequence HAV (i.e., His-Ala-Val), or may also be analogues, peptidomimetics or derivatives thereof.

In one embodiment, particular cadherin antagonists comprise cyclic peptides, or salts thereof, that comprise (1) an intramolecular covalent bond between two non-adjacent residues and (2) at least one classical cadherin cell adhesion recognition (CAR) sequence HAV (His-Ala-Val). The intramolecular bond may be a backbone to backbone, side-chain to backbone or side-chain to side-chain bond (i.e., terminal functional groups of a linear peptide and/or side chain functional groups of a terminal or interior residue may be linked to achieve cyclization). Preferred intramolecular bonds include, but are not limited to, disulfide, amide and thioether bonds. In addition to the classical cadherin CAR sequence HAV, a modulating agent may comprise additional CAR sequences, which may or may not be cadherin CAR sequences, and/or antibodies or fragments thereof that specifically recognize a CAR sequence. Additional CAR sequences may be present within the cyclic peptide containing the HAV sequence, within a separate cyclic peptide component of the modulating agent and/or in a non-cyclic portion of the modulating agent.

Certain preferred HAV-containing cyclic peptides satisfy the formula:

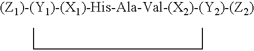

wherein X₁, and X₂ are optional, and if present, are independently selected from the group consisting of amino acid residues and combinations thereof in which the residues are linked by peptide bonds, and wherein X₁ and X₂ independently range in size from 0 to 10 residues, such that the sum of residues contained within X₁ and X₂ ranges from 1 to 12; wherein Y₁ and Y₂ are independently selected from the group consisting of amino acid residues, and wherein a covalent bond is formed between residues Y₁ and Y₂; and wherein Z₁ and Z₂ are optional, and if present, are independently selected from the group consisting of amino acid residues and combinations thereof in which the residues are linked by peptide bonds.

Within certain embodiments, a cyclic peptide may comprise an N-acetyl group (i.e., the amino group present on the amino terminal residue of the peptide prior to cyclization is acetylated) or an N-formyl group (i.e., the amino group present on the amino terminal residue of the peptide prior to cyclization is formylated), or the amino group present on the amino terminal residue of the peptide prior to cyclization is mesylated. One preferred cyclic peptide, for example, is N—Ac-CHAVC-NH₂ (SEQ ID NO:1). Another preferred cyclic peptide is N—Ac-CHAVC-Y—NH₂ (SEQ ID NO:2). Other cyclic peptides include, but are not limited to: N—Ac-CHAVDC-NH₂ (SEQ ID NO:3), N—Ac-CHAVDIC-NH₂ (SEQ ID NO:4), N—Ac-CHAVDINC-NH₂ (SEQ ID NO:5), N—Ac-CHAVDINGC-NH₂ (SEQ ID NO:6), N—Ac-CAHAVC-NH₂ (SEQ ID NO:7), N—Ac-CAHAVDC-NH₂ (SEQ ID NO:8), N—Ac-CAHAVDIC-NH₂ (SEQ ID NO:9), N—Ac-CRAHAVDC-NH₂ (SEQ ID NO:10), N—Ac-CLRAHAVC-NH₂ (SEQ ID NO:11), N—Ac-CLRAHAVDC-NH₂ (SEQ ID NO:12), N—Ac-CSHAVC-NH₂ (SEQ ID NO:13), N—Ac-CFSHAVC-NH₂ (SEQ ID NO:14), N—Ac-CLFSHAVC-NH₂ (SEQ ID NO:15), N—Ac-CHAVSC-NH₂ (SEQ ID NO:16), N—Ac-CSHAVSC-NH₂ (SEQ ID NO:17), N—Ac-CSHAVSSC-NH₂ (SEQ ID NO:18), N—Ac-CHAVSSC-NH₂ (SEQ ID NO:19), N—Ac-KHAVD-NH₂ (SEQ ID NO:20), N—Ac-DHAVK-NH₂ (SEQ ID NO:21), N—Ac-KHAVE-NH₂ (SEQ ID NO:22), N—Ac-AHAVDI-NH₂ (SEQ ID NO:23), N—Ac-SHAVDSS-NH₂ (SEQ ID NO:24), N—Ac-KSHAVSSD-NH₂ (SEQ ID NO:25), N—Ac-CHAVC-S—NH₂ (SEQ ID NO:26), N—Ac—S-CHAVC-NH₂ (SEQ ID NO:27), N—Ac-CHAVC-SS—NH₂ (SEQ ID NO:28), N—Ac—S-CHAVC-S—NH₂ (SEQ ID NO:29), N—Ac-CHAVC-T-NH₂ (SEQ ID NO:30), N—Ac-CHAVC-E-NH₂ (SEQ ID NO:31), N—Ac-CHAVC-D-NH₂ (SEQ ID NO:32), N—Ac-CHAVYC-NH₂ (SEQ ID NO:33), CH₃—SO₂—HN-CHAVC-Y—NH₂ (SEQ ID NO:34), CH₃—SO₂—HN-CHAVC-NH₂ (SEQ ID NO:35), HC(O)—NH-CHAVC-NH₂ (SEQ ID NO:36), N—Ac-CHAVPen-NH₂ (SEQ ID NO:37), N—Ac-PenHAVC-NH₂ (SEQ ID NO:38) and N—Ac-CHAVPC-NH₂ (SEQ ID NO:39).

In addition to CAR sequence(s), cyclic peptides generally comprise at least one additional residue, such that the size of the cyclic peptide ring ranges from 4 to about 15 residues, preferably from 5 to 10 residues. Such additional residue(s) may be present on the N-terminal and/or C-terminal side of a CAR sequence, and may be derived from sequences that flank the HAV sequence within one or more naturally occurring cadherins (e.g., N-cadherin, E-cadherin, P-cadherin, R-cadherin or other cadherins containing the HAV sequence) with or without amino acid substitutions and/or other modifications. Database accession numbers for representative naturally occurring cadherins are as follows: human N-cadherin M34064, mouse N-cadherin M31131 and M22556, cow N-cadherin X53615, human P-cadherin X63629, mouse P-cadherin X06340, human E-cadherin Z13009, mouse E-cadherin X06115. Alternatively, additional residues present on one or both sides of the CAR sequence(s) may be unrelated to an endogenous sequence (e.g., residues that facilitate cyclization).

Within certain embodiments, relatively small cyclic peptides that do not contain significant sequences flanking the HAV sequence are used for modulating N-cadherin and E-cadherin mediated cell adhesion.

b. Cadherin Antagonists Comprising Trp-Containing CAR Sequences

Additional cadherin antagonists useful in the present invention include agents comprising Trp-containing CAR sequences that modulate classical cadherins, as well as peptidomimetics, analogues and derivatives thereof, such as those described in U.S. patent application Ser. No. 10/714,556; US Patent Publication No. 2005/0129676, and PCT Publication No. WO04/044000, the contents of which are incorporated herein by reference in their entireties.

For example, illustrative Trp-containing CAR sequences may comprise the consensus sequence: Asp/Glu-Trp-Val-Ile/Val/Met-Pro/Ala-Pro (SEQ ID NO:40), wherein “Asp/Glu” is an amino acid that is either Asp or Glu, “Ile/Val/Met” is an amino acid that is Ile, Val or Met, and “Pro/Ala” is either Pro or Ala. Particular Trp-containing CAR sequences or conservative analogues thereof include, but are not limited to, DWV, DWVI (SEQ ID NO:41), DWVV (SEQ ID NO: _{—}42), DWVM (SEQ ID NO:43), DWVIP (SEQ ID NO:44), DWVIA (SEQ ID NO:45), DWVVP (SEQ ID NO:46), DWVVPP (SEQ ID NO:47), DWVVAP (SEQ ID NO:48), DWVMPP (SEQ ID NO:49), DWVMAP (SEQ ID NO:50), EWV, EWVI (SEQ ID NO:51), EWVV (SEQ ID NO:52), EWVM (SEQ ID NO:53), EWVIP (SEQ ID NO:54), EWVIA (SEQ ID NO:55), EWVVP (SEQ ID NO:56), EWVVPP (SEQ ID NO:57), EWVVAP (SEQ ID NO:58), EWVMPP (SEQ ID NO:59), EWVMAP (SEQ ID NO:60), WVI, WVIP (SEQ ID NO:61), WVIA (SEQ ID NO:62), WVV, WVVP (SEQ ID NO:63), WVVA (SEQ ID NO:64), WVM, WVMP (SEQ ID NO:65), WVMA (SEQ ID NO:66), WVIPP (SEQ ID NO:67), WVIAP (SEQ ID NO:68), WVVPP (SEQ ID NO:69), WVVAP (SEQ ID NO:70), WVMPP (SEQ ID NO:71), WVMAP (SEQ ID NO:72), DWI, DWII (SEQ ID NO:73), DWIV (SEQ ID NO:74), DWIM (SEQ ID NO:75), DWIIP (SEQ ID NO:76), DWIIA (SEQ ID NO:77), DWIVP (SEQ ID NO:78), DWIVPP (SEQ ID NO:79), DWIVAP (SEQ ID NO:80), DWIMPP (SEQ ID NO:81), DWIMAP (SEQ ID NO:82), EWI, EWII (SEQ ID NO:83), EWIV (SEQ ID NO:84), EWIM (SEQ ID NO:85), EWIIP (SEQ ID NO:86), EWIIA (SEQ ID NO:87), EWIVP (SEQ ID NO:88), EWIVPP (SEQ ID NO:89), EWIVAP (SEQ ID NO:90), EWIMPP (SEQ ID NO:91), EWIMAP (SEQ ID NO:92), WII, WIIP (SEQ ID NO:93), WIIA (SEQ ID NO:94), WIV, WIVP (SEQ ID NO:95), WIVA (SEQ ID NO:96), WIM, WIMP (SEQ ID NO:97), WIMA (SEQ ID NO:98), WIIPP (SEQ ID NO:99), WIIAP (SEQ ID NO:100), WIVPP (SEQ ID NO:101), WIVAP (SEQ ID NO:102), WIMPP (SEQ ID NO:103), WIMAP (SEQ ID NO:104), DWL, DWLI (SEQ ID NO:105), DWLV (SEQ ID NO:106), DWLM (SEQ ID NO:107), DWLIP (SEQ ID NO:108), DWLIA (SEQ ID NO:109), DWLVP (SEQ ID NO: 110), DWLVPP (SEQ ID NO:111), DWLVAP (SEQ ID NO:112), DWLMPP (SEQ ID NO:113), DWLMAP (SEQ ID NO:114), EWL, EWLI (SEQ ID NO:115), EWLV (SEQ ID NO:116), EWLM (SEQ ID NO:117), EWLIP (SEQ ID NO:118), EWLIA (SEQ ID NO:119), EWLVP (SEQ ID NO:120), EWLVPP (SEQ ID NO:121), EWLVAP (SEQ ID NO:122), EWLMPP (SEQ ID NO:123), EWLMAP (SEQ ID NO:124), WLI, WLIP (SEQ ID NO:125), WLIA (SEQ ID NO:126), WLV, WLVP (SEQ ID NO:127), WLVA (SEQ ID NO:128), WLM, WLMP (SEQ ID NO:129), WLMA (SEQ ID NO:130), WLIPP (SEQ ID NO:131), WLIAP (SEQ ID NO:132), WLVPP (SEQ ID NO:133), WLVAP (SEQ ID NO:134), WLMPP (SEQ ID NO:135), WLMAP (SEQ ID NO:136), DWVL (SEQ ID NO:137), DWIL (SEQ ID NO:138), DWLL (SEQ ID NO:139), EWVL (SEQ ID NO:140), EWIL (SEQ ID NO:141), EWLL (SEQ ID NO:142), DWVLP (SEQ ID NO:143), DWILP (SEQ ID NO:144), DWLLP (SEQ ID NO:145), EWVLP (SEQ ID NO:146), EWILP (SEQ ID NO:147), EWLLP (SEQ ID NO:148), DWVLA (SEQ ID NO:149), DWILA (SEQ ID NO:150), DWLLA (SEQ ID NO:151), EWVLA (SEQ ID NO:152), EWILA (SEQ ID NO:153), EWLLA (SEQ ID NO:154), DWVLPP (SEQ ID NO:155), DWILPP (SEQ ID NO:156), DWLLPP (SEQ ID NO:157), EWVLPP (SEQ ID NO:158), EWILPP (SEQ ID NO:159), EWLLPP (SEQ ID NO:160), DWVLAP (SEQ ID NO:161), DWILAP (SEQ ID NO:162), DWLLAP (SEQ ID NO:163), EWVLAP (SEQ ID NO:164), EWILAP (SEQ ID NO:165), EWLLAP (SEQ ID NO:166), WVL, WIL, WLL, WVLP (SEQ ID NO:167), WILP (SEQ ID NO:168), WLLP (SEQ ID NO:169), WVLA (SEQ ID NO:170), WILA (SEQ ID NO:171), WLLA (SEQ ID NO:172), WVLPP (SEQ ID NO:173), WILPP (SEQ ID NO:174), WLLPP (SEQ ID NO:175), WVLAP (SEQ ID NO:176), WILAP (SEQ ID NO:177), and WLLAP (SEQ ID NO:178).

Trp-containing CAR sequences can also be present in cyclic peptide structures, illustrative examples of which may have the following structures:

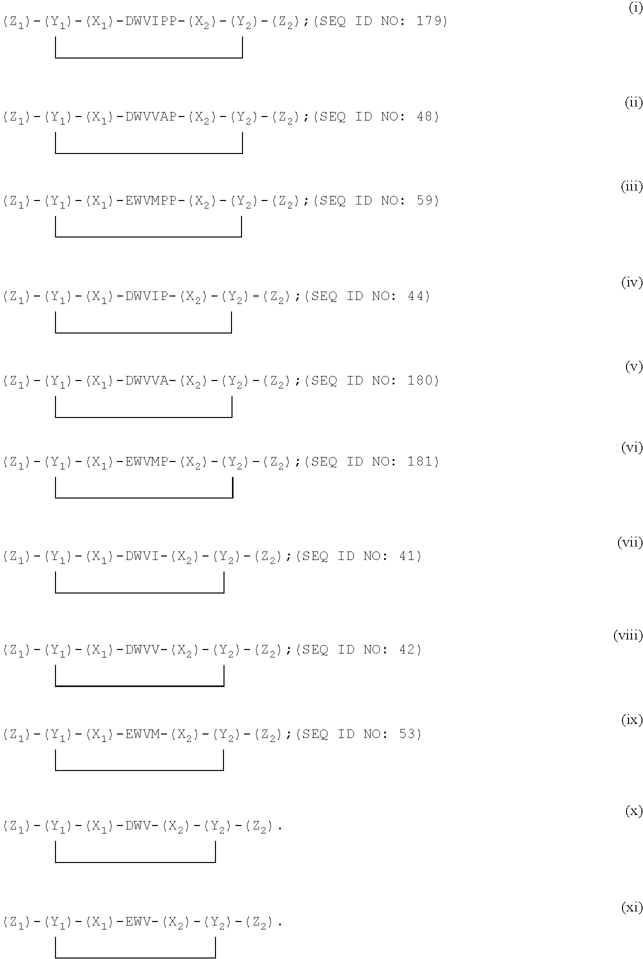

In these structures, X₁ and X₂ are optional, and if present, are amino acid residues or combinations of amino acid residues linked by peptide bonds. X₁ and X₂ may be identical to, or different from, each other. In general, X₁ and X₂ independently range in size from 0 to 10 residues, such that the sum of residues contained within X₁ and X₂ ranges from 1 to 12. Y₁ and Y₂ are amino acid residues, and a covalent bond is formed between residues Y₁ and Y₂. Y₁ and Y₂ may be identical to, or different from, each other. Z₁ and Z₂ are optional, and if present, are amino acid residues or combinations of amino acid residues linked by peptide bonds. Z₁ and Z₂ may be identical to, or different from, each other.

Other cadherin antagonists useful in the present invention include agents comprising Trp-containing CAR sequences that modulate non-classical and atypical cadherins, as well as peptidomimetics, analogues and derivatives thereof, such as those described in US Patent Publication No. 2004/0175361, the content of which is incorporated herein by reference in its entirety.

For example, certain atypical cadherin Trp-containing CAR sequences share the consensus sequence:

(SEQ ID NO: 268)

Gly/Asp/Ser-Trp-Val/Ile/Met-Trp-Asn-Gln

Within the consensus sequence, “Gly/Asp/Ser” indicates an amino acid that is Gly, Asp or Ser; and “Val/Ile/Met” indicates an amino acid that is Val, Ile or Met. Representative atypical cadherin Trp-containing CAR sequences are provided within Table I. Trp-containing CAR sequences specifically provided herein further include portions of such representative Trp-containing CAR sequences, as well as polypeptides that comprise at least a portion of such sequences. Additional atypical cadherin Trp-containing CAR sequences may be identified based on sequence homology to the atypical cadherin Trp-containing CAR sequences provided herein, and based on the ability of a peptide comprising such a sequence to modulate an atypical cadherin-mediated function within a representative assay described herein. Within certain embodiments, an antagonist comprises at least three, four, five and six consecutive residues of an atypical cadherin Trp-containing CAR sequence that satisfies the above consensus sequence.

Exemplary Trp-containing CAR sequences for atypical cadherins include, but are not limited to GWV, GWVW (SEQ ID NO:269), GWVWN (SEQ ID NO:270), GWVWNQ (SEQ ID NO:271), WVW, WVWN (SEQ ID NO:272), WVWNQ (SEQ ID NO:273), DWI, DWIW (SEQ ID NO:274), DWIWN (SEQ ID NO:275), DWIWNQ (SEQ ID NO:276), WIW, WIWN (SEQ ID NO:277), WIWNQ (SEQ ID NO:278), SWM, SWMW (SEQ ID NO:279), SWMWN (SEQ ID NO:280), SWMWNQ (SEQ ID NO:281), WMW, WMWN (SEQ ID NO:282), WMWNQ (SEQ ID NO:283), SWV, SWVW (SEQ ID NO:284), SWVWN (SEQ ID NO:285), SWVWNQ (SEQ ID NO:286), GWM, GWMW (SEQ ID NO:287), GWMWN (SEQ ID NO:288), GWMWNQ (SEQ ID NO:289), AWV, AWVI (SEQ ID NO:290), AWVIP (SEQ ID NO:291), AWVIPP (SEQ ID NO:292), WVI, WVIP (SEQ ID NO:293), WVIPP (SEQ ID NO:294), GWVWNQF (SEQ ID NO:295), GWVWNQFF (SEQ ID NO:296), GWVWNQFFV (SEQ ID NO:297), WVWNQF (SEQ ID NO:298), WVWNQFF (SEQ ID NO:299), WVWNQFFV (SEQ ID NO:300), RGW, RGWV (SEQ ID NO:301), RGWVW (SEQ ID NO:302), RGWVWN (SEQ ID NO:303), RGWVWNQ (SEQ ID NO:304), RGWVWNQF (SEQ ID NO:305), RGWVWNQFF (SEQ ID NO:306), RGWVWNQFFV (SEQ ID NO:307), KRGW (SEQ ID NO:308), KRGWV (SEQ ID NO:309), KRGWVW (SEQ ID NO:310), KRGWVWN (SEQ ID NO:311), KRGWVWNQ (SEQ ID NO:312), KRGWVWNQF (SEQ ID NO:313), KRGWVWNQFF (SEQ ID NO:314), KRGWVWNQFFV (SEQ ID NO:315), DWIWNQM (SEQ ID NO:316), DWIWNQMH (SEQ ID NO:317), DWIWNQMHI (SEQ ID NO:318), WIWNQM (SEQ ID NO:319), WIWNQMH (SEQ ID NO:320), WIWNQMHI (SEQ ID NO:321), RDW, RDWI (SEQ ID NO:322), RDWIW (SEQ ID NO:323), RDWIWN (SEQ ID NO:324), RDWIWNQ (SEQ ID NO:325), RDWIWNQM (SEQ ID NO:326), RDWIWNQMH (SEQ ID NO:327), RDWIWNQMHI (SEQ ID NO:328), KRDW (SEQ ID NO:329), KRDWI (SEQ ID NO:330), KRDWIW (SEQ ID NO:331), KRDWIWN (SEQ ID NO:332), KRDWIWNQ (SEQ ID NO:333), KRDWIWNQM (SEQ ID NO:334), KRDWIWNQMH (SEQ ID NO:335), KRDWIWNQMHI (SEQ ID NO:336), SWMWNQF (SEQ ID NO:337), SWMWNQFF (SEQ ID NO:338), SWMWNQFFL (SEQ ID NO:339), WMWNQF (SEQ ID NO:340), WMWNQFF (SEQ ID NO:341), WMWNQFFL (SEQ ID NO:342), RSW, RSWM (SEQ ID NO:343), RSWMW (SEQ ID NO:344), RSWMWN (SEQ ID NO:345), RSWMWNQ (SEQ ID NO:346), RSWMWNQF (SEQ ID NO:347), RSWMWNQFF (SEQ ID NO:348), RSWMWNQFFL (SEQ ID NO:349), KRSW (SEQ ID NO:350), KRSWM (SEQ ID NO:351), KRSWMW (SEQ ID NO:352), KRSWMWN (SEQ ID NO:353), KRSWMWNQ (SEQ ID NO:354), KRSWMWNQF (SEQ ID NO:355), KRSWMWNQFF (SEQ ID NO:356), KRSWMWNQFFL (SEQ ID NO:357), SWVWNQF (SEQ ID NO:358), SWVWNQFF (SEQ ID NO:359), SWVWNQFFV (SEQ ID NO:360), WVWNQF (SEQ ID NO:361), WVWNQFF (SEQ ID NO:362), WVWNQFFV (SEQ ID NO:363), RSWV (SEQ ID NO:364), RSWVW (SEQ ID NO:365), RSWVWN (SEQ ID NO:366), RSWVWNQ (SEQ ID NO:367), RSWVWNQF (SEQ ID NO:368), RSWVWNQFF (SEQ ID NO:369), RSWVWNQFFV (SEQ ID NO:370), KRSWV (SEQ ID NO:371), KRSWVW (SEQ ID NO:372), KRSWVWN (SEQ ID NO:373), KRSWVWNQ (SEQ ID NO:374), KRSWVWNQF (SEQ ID NO:375), KRSWVWNQFF (SEQ ID NO:376), KRSWVWNQFFV (SEQ ID NO:377), GWVWNQM (SEQ ID NO:378), GWVWNQMF (SEQ ID NO:379), GWVWNQMFV (SEQ ID NO:380), RGWVWNQM (SEQ ID NO:381), RGWVWNQMF (SEQ ID NO:382), RGWVWNQMFV (SEQ ID NO:383), KRGWVWNQM (SEQ ID NO:384), KRGWVWNQMFV (SEQ ID NO:385), GWVWNQFFL (SEQ ID NO:386), RGWVWNQFFL (SEQ ID NO:387), KRGWVWNQFFL (SEQ ID NO:388), AWVIPPI (SEQ ID NO:389), AWVIPPIS (SEQ ID NO:390), AWVIPPISV (SEQ ID NO:391), WVIPPI (SEQ ID NO:392), WVIPPIS (SEQ ID NO:393), WVIPPISV (SEQ ID NO:394), RAW, RAWV (SEQ ID NO:395), RAWVI (SEQ ID NO:396), RAWVIP (SEQ ID NO:397), RAWVIPP (SEQ ID NO:398), RAWVIPPI (SEQ ID NO:399), RAUVIPPIS (SEQ ID NO:400), RAWVIPPISV (SEQ ID NO:401), KRAW (SEQ ID NO:402), KRAWV (SEQ ID NO:403), KRAWVI (SEQ ID NO:404), KRAWVIP (SEQ ID NO:405), KRAWVIPP (SEQ ID NO:406), KRAWVIPPI (SEQ ID NO:407), KRAWVIPPIS (SEQ ID NO:408), VWN, VWNQ (SEQ ID NO:409), VWNQM (SEQ ID NO:410), VWNQF (SEQ ID NO:411), VWNQMF (SEQ ID NO:412), VWNQFF (SEQ ID NO:413), WNQ, WNQM (SEQ ID NO:414), WNQF (SEQ ID NO:415), WNQFF (SEQ ID NO:416), IWN, IWNQ (SEQ ID NO:417), IWNQM (SEQ ID NO:418), IWNQMH (SEQ ID NO:419), WNQM (SEQ ID NO:420), WNQMH (SEQ ID NO:421), MWN, MWNQ (SEQ ID NO:422), MWNQF (SEQ ID NO:423), and MWNQFF (SEQ ID NO:424).

Other atypical cadherin antagonists are present within a cyclic peptide ring comprising the sequence G/S/D-W-V/M/I-W-N-Q (SEQ ID NO:268), the sequence AWVIPP (SEQ ID NO:292), or a portion thereof. Exemplary cyclic peptides have the following formula:

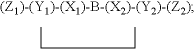

In this formula, B represents an amino acid sequence selected from the following sequences: DWIWNQ (SEQ ID NO:276), SWMWNQ (SEQ ID NO:281), SWVWNQ (SEQ ID NO:286), GWVWNQ (SEQ ID NO:271), AWVIPP (SEQ ID NO:292), GWVWN (SEQ ID NO:270), DWIWN (SEQ ID NO:275), SWMWN (SEQ ID NO:280), SWVWN (SEQ ID NO:285), GWVWN (SEQ ID NO:270), AWVIP (SEQ ID NO:291), GWVW (SEQ ID NO:269), DWIW (SEQ ID NO:274), SWMW (SEQ ID NO:279), SWVW (SEQ ID NO:284), GWVW (SEQ ID NO:269), AWVI (SEQ ID NO:290), GWV, DWI, SWM, SWV, GWV, AWV, VWN, VWNQ (SEQ ID NO:409), VWNQM (SEQ ID NO:410), VWNQF (SEQ ID NO:411), VWNQMF (SEQ ID NO:412), VWNQFF (SEQ ID NO:413), WNQ, WNQM (SEQ ID NO:414), WNQF (SEQ ID NO:415), WNQFF (SEQ ID NO:416), IWN, IWNQ (SEQ ID NO:417), IWNQM (SEQ ID NO:418), IWNQMH (SEQ ID NO:419), WNQM (SEQ ID NO:420), WNQMH (SEQ ID NO:421), MWN, MWNQ (SEQ ID NO:422), MWNQF (SEQ ID NO:423), and MWNQFF (SEQ ID NO:424). X₁ and X₂ are optional, and if present, are amino acid residues or combinations of amino acid residues linked by peptide bonds. X₁ and X₂ may be identical to, or different from, each other. In general, X₁ and X₂ independently range in size from 0 to 10 residues, such that the sum of residues contained within X₁ and X₂ ranges from 1 to 12. Y₁ and Y₂ are amino acid residues, and a covalent bond is formed between residues Y₁ and Y₂. Y₁ and Y₂ may be identical to, or different from, each other. Z₁ and Z₂ are optional, and if present, are amino acid residues or combinations of amino acid residues linked by peptide bonds. Z₁ and Z₂ may be identical to, or different from, each other.
c. Cadherin Antagonists Comprising HAV-BM CAR Sequences

Other cadherin antagonists for use in the invention comprise compounds referred to as HAV-binding motif (HAV-BM) sequences, such as those described, e.g., in U.S. Pat. Nos. 6,277,824; 6,472,368; and 6,806,255. Such agents generally comprise an HAV-BM sequence, or an analogue, peptidomimetic or derivative thereof. In a particular embodiment, the HAV-BM sequence comprises the sequence: (a) Ile/Val-Phe-Aaa-Ile-Baa-Caa-Daa-Ser/Thr-Gly-Eaa-Leu/Met (SEQ ID NO:182), wherein Aaa, Baa, Caa, Daa and Eaa are independently selected from the group consisting of amino acid residues; or comprises the sequence Trp-Leu-Aaa-Ile-Asp/Asn-Baa-Caa-Daa-Gly-Gln-Ile (SEQ ID NO:183), wherein Aaa, Baa, Caa and Daa are independently selected from the group consisting of amino acid residues.

Certain illustrative HAV-BM sequences include, but are not limited to, sequences selected from the group consisting of: IFIINPISGQL (SEQ ID NO:184), IFILNPISGQL (SEQ ID NO:185), VFAVEKETGWL (SEQ ID NO:186), VFSINSMSGRM (SEQ ID NO:187), VFIIERETGWL (SEQ ID NO:188), VFTIEKESGWL (SEQ ID NO:189), VFNIDSMSGRM (SEQ ID NO:190), WLKIDSVNGQI (SEQ ID NO:191), WLKIDPVNGQI (SEQ ID NO:192), WLAMDPDSGQV (SEQ ID NO:193), WLHINATNGQI (SEQ ID NO:194), WLEINPDTGAI (SEQ ID NO:195), WLAVDPDSGQI (SEQ ID NO:196), WLEINPETGAI (SEQ ID NO:197), WLHINTSNGQI (SEQ ID NO:198), NLKIDPVNGQI (SEQ ID NO:199), LKIDPVNGQI (SEQ ID NO:200) and analogues of the foregoing sequences that retain at least seven consecutive residues (e.g., INPISGQ (SEQ ID NO:201), LNPISGQ (SEQ ID NO:202), IDPVSGQ (SEQ ID NO:203) or KIDPVNGQ (SEQ ID NO:204)), wherein the ability of the analogue to modulate a cadherin-mediated process is not diminished. Alternatively, an agent may be an HAV-BM sequence that comprises at least five consecutive residues of a peptide selected from the group consisting of INPISGQ (SEQ ID NO:201), LNPISGQ (SEQ ID NO:202), NLKIDPVNGQI (SEQ ID NO:203) and WLKIDPVNGQI (SEQ ID NO:204). For example, the agent may comprise a sequence selected from the group consisting of PISGQ (SEQ ID NO:205), PVNGQ (SEQ ID NO:206), PVSGR (SEQ ID NO:207), IDPVN (SEQ ID NO:208), INPIS (SEQ ID NO:209) and KIDPV (SEQ ID NO:210).

An HAV-BM sequence may be present within a linear peptide or a cyclic peptide. Certain illustrative cyclic peptides include, but are not limited to, the following structures:

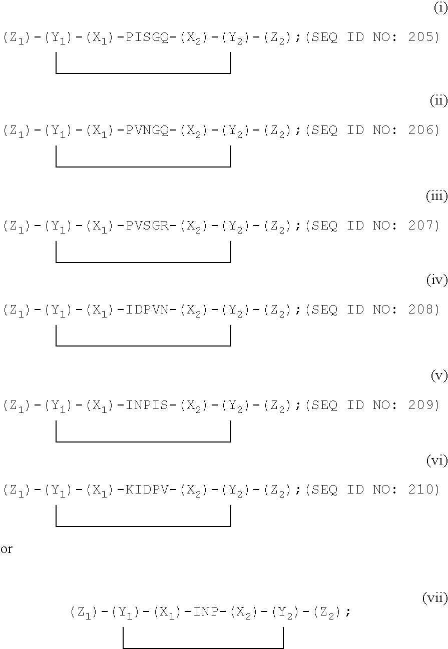

wherein X₁, and X₂ are optional, and if present, are independently selected from the group consisting of amino acid residues and combinations thereof in which the residues are linked by peptide bonds, and wherein X₁ and X₂ independently range in size from 0 to 10 residues, such that the sum of residues contained within X₁ and X₂ ranges from 1 to 12; wherein Y₁ and Y₂ are independently selected from the group consisting of amino acid residues, and wherein a covalent bond is formed between residues Y₁ and Y₂; and wherein Z₁ and Z₂ are optional, and if present, are independently selected from the group consisting of amino acid residues and combinations thereof in which the residues are linked by peptide bonds. Such cyclic peptides may contain modifications. For example, Y₁ may comprise an N-acetyl group and/or Y₂ may comprise a C-terminal amide group. Cyclization may be achieved in any of a variety of ways, such as covalent linkage of Y₁ and Y₂ via a disulfide, amide or thioether bond.

In addition to the illustrative peptide-based CAR sequences and structures discussed herein, suitable cadherin antagonists for use in the invention may also comprise analogues, peptidomimetics and derivatives thereof, as discussed herein and in the references incorporated herein.

d. Antibody-Based Cadherin Antagonists

Other illustrative cadherin antagonists used in the invention may comprise antibodies, or antigen-binding fragments thereof, that are capable of modulating one or more cadherin-mediated processes or functions. For example, antibodies, and antigen-binding fragments thereof, may include those that specifically bind to a region of a cadherin and as a result antagonize one or more functions or processes mediated by the cadherin, such as cell adhesion. Particular antibodies, and antigen-binding fragments thereof, effective as cadherin antagonists, include antibodies capable of binding one or more CAR sequences described above and/or described in one or more of the references incorporated by reference herein (e.g., U.S. Pat. Nos. 6,031,072; 6,417,325; 6,465,427; 6,780,845; 6,203,788; WO05/012348; U.S. patent application Ser. No. 10/714,556; US Patent Publication Nos. 2005/0129676, 2005/0215482, 2005/0222037, 2005/0203025, 2004/0175361, PCT Publication No. WO04/044000; U.S. Pat. Nos. 6,277,824; 6,472,368; and 6,806,255).

An antibody, or antigen-binding fragment thereof, is said to “specifically bind” to a cadherin sequence (with or without flanking amino acids) if it reacts at a detectable level (within, for example, an ELISA, as described by Newton et al., Develop. Dynamics 197:1-13, 1993) with a peptide containing that sequence, and does not react at a detectable level, within the same or similar assay, with peptides containing a different sequence or a sequence in which the order of amino acid residues in the sequence and/or flanking sequence is different or has been altered.

Antibodies and fragments thereof may be prepared using standard techniques. See, e.g., Harlow and Lane, Antibodies: A Laboratory Manual, Cold Spring Harbor Laboratory, 1988. In one such technique, an immunogen comprising a CAR sequence is initially injected into any of a wide variety of mammals (e.g., mice, rats, rabbits, sheep or goats). Small immunogens (i.e., less than about 20 amino acids) should be joined to a carrier protein, such as bovine serum albumin or keyhole limpet hemocyanin. Following one or more injections, the animals are bled periodically. Polyclonal antibodies specific for the CAR sequence may then be purified from such antisera by, for example, affinity chromatography using the modulating agent or antigenic portion thereof coupled to a suitable solid support.

Monoclonal antibodies specific for a cadherin sequence may be prepared, for example, using the technique of Kohler and Milstein, Eur. J. Immunol. 6:511-519, 1976, and improvements thereto. Briefly, these methods involve the preparation of immortal cell lines capable of producing antibodies having the desired specificity from spleen cells obtained from an animal immunized as described above. The spleen cells are immortalized by, for example, fusion with a myeloma cell fusion partner, preferably one that is syngeneic with the immunized animal. Single colonies are selected and their culture supernatants tested for binding activity against the modulating agent or antigenic portion thereof. Hybridomas having high reactivity and specificity are preferred.

Monoclonal antibodies may be isolated from the supernatants of growing hybridoma colonies, with or without the use of various techniques known in the art to enhance the yield. Contaminants may be removed from the antibodies by conventional techniques, such as chromatography, gel filtration, precipitation, and extraction. Antibodies having the desired activity may generally be identified using immunofluorescence analyses of tissue sections, cell or other samples where the target cadherin is localized.

Within certain embodiments, antigen-binding fragments of antibodies are employed. Such fragments include Fab fragments, which may be prepared using standard techniques. Briefly, immunoglobulins may be purified from rabbit serum by affinity chromatography on Protein A bead columns (Harlow and Lane, Antibodies: A Laboratory Manual, Cold Spring Harbor Laboratory, 1988; see especially page 309) and digested by papain to yield Fab and Fc fragments. The Fab and Fc fragments may be separated by affinity chromatography on protein A bead columns (Harlow and Lane, 1988, pages 628-29).

e. Peptidomimetic & Small Molecule-Based N-Cadherin Antagonists

Still further cadherin antagonists useful in the methods of the invention include peptidomimetics and small molecules having a three-dimensional structure that is substantially similar to a three-dimensional structure of a cyclic peptide antagonist that comprises the CAR sequence HAV within a cyclic peptide ring, such as those described in U.S. patent application Ser. No. 10/412,701 and PCT Publication No. WO01/53331, the contents of which are incorporated herein by reference in their entireties.

f. Other Cadherin Antagonists

Other cadherin antagonists useful in the present invention include, for example, those capable of modulating non-classical cadherins, such as OB-cadherin and VE-cadherin. Illustrative non-classical cadherin antagonists include, for example, those described in US Patent Publication Nos. 2005/0215482; 2005/0222037; and 2005/0203025, the contents of which are incorporated herein by reference in their entireties.

Illustrative examples of non-classical cadherin CAR sequence have the formula:

(SEQ ID NO: 211)

Aaa-Phe-Baa-Ile/Leu/Val-Asp/Asn/Glu-Caa-Daa-
Ser/Thr/Asn-Gly

wherein Aaa, Baa, Caa and Daa are independently selected amino acid residues; Ile/Leu/Val is an amino acid that is selected from the group consisting of isoleucine, leucine and valine, Asp/Asn/Glu is an amino acid that is selected from the group consisting of aspartate, asparagine and glutamate; and Ser/Thr/Asn is an amino acid that is selected from the group consisting of serine, threonine or asparagine. For other antagonists as described, the non-classical cadherin CAR sequence consists of at least three consecutive amino acid residues, and preferably at least five consecutive amino acid residues, of a non-classical cadherin, wherein the consecutive amino acids are present within a region of the non-classical cadherin having the formula recited above. Other agents may comprise at least nine consecutive amino acid residues of a non-classical cadherin, wherein the nine consecutive amino acid residues comprise a region having a formula as recited above.

Within certain specific embodiments, an antagonist is a peptide ranging in size from 3 to 50, preferably from 4 to 16, amino acid residues.

Within other embodiments, an antagonist comprises a non-classical cadherin CAR sequence that is present within a cyclic peptide. Such cyclic peptides may have the formula:

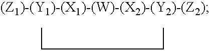

wherein W is a tripeptide selected from the group consisting of EEY, DDK, EAQ, DAE, NEN, ESE, DSG, DEN, EPK, DAN, EEF, NDV, DET, DPK, DDT, DAN, DKF, DEL, DAD, NNK, DLV, NRD, DPS, NQK, NRN, NKD, EKD, ERD, DPV, DSV, DLY, DSN, DSS, DEK, NEK; RAL, YAL, YAT, FAT and YAS wherein X₁, and X₂ are optional, and if present, are independently selected from the group consisting of amino acid residues and combinations thereof in which the residues are linked by peptide bonds, and wherein X₁ and X₂ independently range in size from 0 to 10 residues, such that the sum of residues contained within X₁ and X₂ ranges from 1 to 12; wherein Y₁ and Y₂ are independently selected from the group consisting of amino acid residues, and wherein a covalent bond is formed between residues Y₁ and Y₂; and wherein Z₁ and Z₂ are optional, and if present, are independently selected from the group consisting of amino acid residues and combinations thereof in which the residues are linked by peptide bonds.

The present invention also employs antagonists that comprise an antibody or antigen-binding fragment thereof that specifically binds to a non-classical cadherin CAR sequence and modulates a non-classical cadherin-mediated function,

Within further aspects, the present invention employs antagonists comprising a non-peptide mimetic of any one of the non-classical cadherin CAR sequences provided above and/or in the references incorporated herein.

Certain illustrative OB-cadherin antagonists comprise: (a) one or more OB-cadherin CAR sequences selected from the group consisting of DDK, IDDK (SEQ ID NO:212) DDKS (SEQ ID NO:213), VIDDK (SEQ ID NO:214), IDDKS (SEQ ID NO:215), VIDDKS (SEQ ID NO:216), DDKSG (SEQ ID NO:217), IDDKSG (SEQ ID NO:218), VIDDKSG (SEQ ID NO:219), FVIDDK (SEQ ID NO:220), FVIDDKS (SEQ ID NO:221), FVIDDKSG (SEQ ID NO:222), IFVIDDK (SEQ ID NO:223), IFVIDDKS (SEQ ID NO:224), IFVIDDKSG (SEQ ID NO:225), EEY, IEEY (SEQ ID NO:226), EEYT (SEQ ID NO:227), VIEEY (SEQ ID NO:228), IEEYT (SEQ ID NO:229), VIEEYT (SEQ ID NO:230), EEYTG (SEQ ID NO:231), IEEYTG (SEQ ID NO:232), VIEEYTG (SEQ ID NO:233), FVIEEY (SEQ ID NO:234), FVIEEYT (SEQ ID NO:235), FVIEEYTG (SEQ ID NO:236), FFVIEEY (SEQ ID NO:237), FFVIEEYT (SEQ ID NO:238), FFVIEEYTG (SEQ ID NO:239), EAQ, VEAQ (SEQ ID NO:240), EAQT (SEQ ID NO:241), SVEAQ (SEQ ID NO:242), VEAQT (SEQ ID NO:243), SVEAQT (SEQ ID NO:244), EAQTG (SEQ ID NO:245), VEAQTG (SEQ ID NO:246), SVEAQTG (SEQ ID NO:247), FSVEAQ (SEQ ID NO:248), FSVEAQT (SEQ ID NO:249), FSVEAQTG (SEQ ID NO:250), YFSVEAQ (SEQ ID NO:251), YFSVEAQT (SEQ ID NO:252) and YFSVEAQTG (SEQ ID NO:253); or (b) an analogue of any of the foregoing sequences that differs in one or more substitutions, deletions, additions and/or insertions such that that ability of the analogue to modulate an OB-cadherin-mediated function is not substantially diminished. For example, the agent may comprise a linear peptide having the sequence N—Ac-IFVIDDKSG-NH₂ (SEQ ID NO:225), N—Ac-FFVIEEYTG-NH₂ (SEQ ID NO:239) or N—Ac-YFSVEAQTG-NH₂ (SEQ ID NO:253). The OB-cadherin CAR sequence may, but need not, be present within a cyclic peptide.

Illustrative cadherin-5 (also known as VE-cadherin) antagonists can comprise: (a) one or more cadherin-5 CAR sequences selected from the group consisting of DAE, VDAE (SEQ ID NO:254), DAET (SEQ ID NO:255), RVDAE (SEQ ID NO:256), VDAET (SEQ ID NO:257), RVDAET (SEQ ID NO:258), DAETG (SEQ ID NO:259), VDAETG (SEQ ID NO:260), RVDAETG (SEQ ID NO:261), FRVDAE (SEQ ID NO:262), FRVDAET (SEQ ID NO:263), FRVDAETG (SEQ ID NO:264), VFRVDAE (SEQ ID NO:265), VFRVDAET (SEQ ID NO:266) and VFRVDAETG (SEQ ID NO:267); or (b) an analogue of any of the foregoing sequences that differs in one or more substitutions, deletions, additions and/or insertions such that that ability of the analogue to modulate a cadherin-5-mediated function is not substantially diminished. For example, the agent may comprise a linear peptide having the sequence N—Ac-VFRVDAETG-NH₂ (SEQ ID NO:267). The cadherin-5 CAR sequence may, but need not, be present within a cyclic peptide.

g. Preparation of Cadherin Antagonists

The preparation and characterization of linear and/or cyclic peptide antagonists is well known and is illustratively described in the references incorporated herein. For example, for certain embodiments, to facilitate the preparation of cyclic peptides having a desired specificity, nuclear magnetic resonance (NMR) and computational techniques may be used to determine the conformation of a peptide that confers a known specificity. NMR is widely used for structural analysis of molecules. Cross-peak intensities in nuclear Overhauser enhancement (NOE) spectra, coupling constants and chemical shifts depend on the conformation of a compound. NOE data provide the interproton distance between protons through space and across the ring of the cyclic peptide. This information may be used to facilitate calculation of the low energy conformations for the CAR sequence. Conformation may then be correlated with tissue specificity to permit the identification of peptides that are similarly tissue specific or have enhanced tissue specificity.

Cyclic peptides as described herein may comprise residues of L-amino acids, D-amino acids, or any combination thereof. Amino acids may be from natural or non-natural sources, provided that at least one amino group and at least one carboxyl group are present in the molecule; α- and β-amino acids are generally preferred. The 20 L-amino acids commonly found in proteins are identified herein by the conventional three-letter or one-letter abbreviations indicated in Table 1, and the corresponding D-amino acids are designated by a lower case one letter symbol. Modulating agents and cyclic peptides may also contain one or more rare amino acids (such as 4-hydroxyproline or hydroxylysine), organic acids or amides and/or derivatives of common amino acids, such as amino acids having the C-terminal carboxylate esterified (e.g., benzyl, methyl or ethyl ester) or amidated and/or having modifications of the N-terminal amino group (e.g., acetylation or alkoxycarbonylation), with or without any of a wide variety of side-chain modifications and/or substitutions (e.g., methylation, benzylation, t-butylation, tosylation, alkoxycarbonylation, and the like). Certain derivatives include amino acids having an N-acetyl group (such that the amino group that represents the N-terminus of the linear peptide prior to cyclization is acetylated) and/or a C-terminal amide group (i.e., the carboxy terminus of the linear peptide prior to cyclization is amidated). Residues other than common amino acids that may be present with a cyclic peptide include, but are not limited to, penicillamine, β,β-tetramethylene cysteine, β,β-pentamethylene cysteine, β-mercaptopropionicacid, β,β-pentamethylene-β-mercaptopropionic acid, 2-mercaptobenzene, 2-mercaptoaniline, 2-mercaptoproline, ornithine, diaminobutyric acid, β-aminoadipic acid, m-aminomethylbenzoic acid and α,β-diaminopropionic acid.

TABLE 1
Amino acid one-letter and three-letter abbreviations

| | | |
|---|---|---|
| A | Ala | Alanine |
| R | Arg | Arginine |
| D | Asp | Aspartic acid |
| N | Asn | Asparagine |
| C | Cys | Cysteine |
| Q | Gln | Glutamine |
| E | Glu | Glutamic acid |
| G | Gly | Glycine |
| H | His | Histidine |
| I | Ile | Isoleucine |
| L | Leu | Leucine |
| K | Lys | Lysine |
| M | Met | Methionine |
| F | Phe | Phenylalanine |
| P | Pro | Proline |
| S | Ser | Serine |
| T | Thr | Threonine |
| W | Trp | Tryptophan |
| Y | Tyr | Tyrosine |
| V | Val | Valine |

Cyclic peptides as described herein may be synthesized by methods well known in the art, including recombinant DNA methods and chemical synthesis. Chemical synthesis may generally be performed using standard solution phase or solid phase peptide synthesis techniques, in which a peptide linkage occurs through the direct condensation of the α-amino group of one amino acid with the α-carboxy group of the other amino acid with the elimination of a water molecule. Peptide bond synthesis by direct condensation, as formulated above, requires suppression of the reactive character of the amino group of the first and of the carboxyl group of the second amino acid. The masking substituents must permit their ready removal, without inducing breakdown of the labile peptide molecule.

In solution phase synthesis, a wide variety of coupling methods and protecting groups may be used (see Gross and Meienhofer, eds., “The Peptides: Analysis, Synthesis, Biology,” Vol. 1-4 (Academic Press, 1979); Bodansky and Bodansky, “The Practice of Peptide Synthesis,” 2d ed. (Springer Verlag, 1994)). In addition, intermediate purification and linear scale up are possible. Those of ordinary skill in the art will appreciate that solid phase and solution synthesis requires consideration of main chain and side chain protecting groups and activation method. In addition, careful segment selection is necessary to minimize racemization during segment condensation. Solubility considerations are also a factor.

Solid phase peptide synthesis uses an insoluble polymer for support during organic synthesis. The polymer-supported peptide chain permits the use of simple washing and filtration steps instead of laborious purifications at intermediate steps. Solid-phase peptide synthesis may generally be performed according to the method of Merrifield et al., J. Am. Chem. Soc. 85:2149, 1963, which involves assembling a linear peptide chain on a resin support using protected amino acids. Solid phase peptide synthesis typically utilizes either the Boc or Fmoc strategy. The Boc strategy uses a 1% cross-linked polystyrene resin. The standard protecting group for α-amino functions is the tert-butyloxycarbonyl (Boc) group. This group can be removed with dilute solutions of strong acids such as 25% trifluoroacetic acid (TFA). The next Boc-amino acid is typically coupled to the amino acyl resin using dicyclohexylcarbodiimide (DCC). Following completion of the assembly, the peptide-resin is treated with anhydrous HF to cleave the benzyl ester link and liberate the free peptide. Side-chain functional groups are usually blocked during synthesis by benzyl-derived blocking groups, which are also cleaved by HF. The free peptide is then extracted from the resin with a suitable solvent, purified and characterized. Newly synthesized peptides can be purified, for example, by gel filtration, HPLC, partition chromatography and/or ion-exchange chromatography, and may be characterized by, for example, mass spectrometry or amino acid sequence analysis. In the Boc strategy, C-terminal amidated peptides can be obtained using benzhydrylamine or methylbenzhydrylamine resins, which yield peptide amides directly upon cleavage with HF.

In the procedures discussed above, the selectivity of the side-chain blocking groups and of the peptide-resin link depends upon the differences in the rate of acidolytic cleavage. Orthogonal systems have been introduced in which the side-chain blocking groups and the peptide-resin link are completely stable to the reagent used to remove the α-protecting group at each step of the synthesis. The most common of these methods involves the 9-fluorenylmethyloxycarbonyl (Fmoc) approach. Within this method, the side-chain protecting groups and the peptide-resin link are completely stable to the secondary amines used for cleaving the N-α-Fmoc group. The side-chain protection and the peptide-resin link are cleaved by mild acidolysis. The repeated contact with base makes the Merrifield resin unsuitable for Fmoc chemistry, and p-alkoxybenzyl esters linked to the resin are generally used. Deprotection and cleavage are generally accomplished using TFA.

Those of ordinary skill in the art will recognize that, in solid phase synthesis, deprotection and coupling reactions must go to completion and the side-chain blocking groups must be stable throughout the entire synthesis. In addition, solid phase synthesis is generally most suitable when peptides are to be made on a small scale.

Acetylation of an N-terminal residue can be accomplished, for example, by reacting the final peptide with acetic anhydride before cleavage from the resin. C-amidation is accomplished using an appropriate resin such as methylbenzhydrylamine resin using the Boc technology.

Following synthesis of a linear peptide, with or without N-acetylation and/or C-amidation, cyclization may be achieved by any of a variety of techniques well known in the art. Within one embodiment, a bond may be generated between reactive amino acid side chains. For example, a disulfide bridge may be formed from a linear peptide comprising two thiol-containing residues by oxidizing the peptide using any of a variety of methods. Within one such method, air oxidation of thiols can generate disulfide linkages over a period of several days using either basic or neutral aqueous media. The peptide is used in high dilution to minimize aggregation and intermolecular side reactions. This method suffers from the disadvantage of being slow but has the advantage of only producing H₂O as a side product. Alternatively, strong oxidizing agents such as 12 and K₃Fe(CN)₆ can be used to form disulfide linkages. Those of ordinary skill in the art will recognize that care must be taken not to oxidize the sensitive side chains of Met, Tyr, Trp or His. Cyclic peptides produced by this method require purification using standard techniques, but this oxidation is applicable at acid pHs.

Oxidizing agents also allow concurrent deprotection/oxidation of suitable S-protected linear precursors to avoid premature, nonspecific oxidation of free cysteine. DMSO, unlike 12 and K₃Fe(CN)₆, is a mild oxidizing agent which does not cause oxidative side reactions of the nucleophilic amino acids mentioned above. DMSO is miscible with H₂O at all concentrations, and oxidations can be performed at acidic to neutral pHs with harmless byproducts. Methyltrichlorosilane-diphenylsulfoxide may alternatively be used as an oxidizing agent, for concurrent deprotection/oxidation of S-Acm, S-Tacm or S-t-Bu of cysteine without affecting other nucleophilic amino acids. There are no polymeric products resulting from intermolecular disulfide bond formation.

Suitable thiol-containing residues for use in such oxidation methods include, but are not limited to, cysteine, β,β-dimethyl cysteine (penicillamine or Pen), β,β-tetramethylene cysteine (Tmc), β,β-pentamethylene cysteine (Pmc), β-mercaptopropionic acid (Mpr), β,β-pentamethylene-β-mercaptopropionic acid (Pmp), 2-mercaptobenzene, 2-mercaptoaniline and 2-mercaptoproline.

As noted above, a modulating agent may consist entirely of one or more cyclic peptides, or may contain additional peptide and/or non-peptide sequences. Peptide portions may be synthesized as described above or may be prepared using recombinant methods. Within such methods, all or part of a modulating agent can be synthesized in living cells, using any of a variety of expression vectors known to those of ordinary skill in the art to be appropriate for the particular host cell. Suitable host cells may include bacteria, yeast cells, mammalian cells, insect cells, plant cells, algae and other animal cells (e.g., hybridoma, CHO, myeloma). The DNA sequences expressed in this manner may encode portions of an endogenous cadherin or other adhesion molecule. Such sequences may be prepared based on known cDNA or genomic sequences (see Blaschuk et al., J. Mol. Biol. 211:679-682, 1990), or from sequences isolated by screening an appropriate library with probes designed based on the sequences of known cadherins. Such screens may generally be performed as described in Sambrook et al., Molecular Cloning: A Laboratory Manual, Cold Spring Harbor Laboratories, Cold Spring Harbor, N.Y., 1989 (and references cited therein). Polymerase chain reaction (PCR) may also be employed, using oligonucleotide primers in methods well known in the art, to isolate nucleic acid molecules encoding all or a portion of an endogenous adhesion molecule. To generate a nucleic acid molecule encoding a peptide portion of a modulating agent, an endogenous sequence may be modified using well known techniques. For example, portions encoding one or more CAR sequences may be joined, with or without separation by nucleic acid regions encoding linkers, as discussed above. Alternatively, portions of the desired nucleic acid sequences may be synthesized using well known techniques, and then ligated together to form a sequence encoding a portion of the modulating agent.

2. Anticancer Agents and Treatments Used in Combination with Cadherin Antagonists

As noted above, the present invention provides improved therapeutic benefit in the context of cancer treatment methods when cadherin antagonists are used in combination with certain anticancer agents.

In one embodiment, anticancer agents used in combination with cadherin antagonists may comprise anticancer alkylating agents, including, but not limited to: (1) nitrogen mustards (e.g., mechlorethamine, cyclophosphamide, ifosfamide, trofosfamide, melphalan (L-sarcolysin) and chlorambucil); (2) ethylenimines and methylmelamines (e.g., hexamethylmelamine and thiotepa); (3) alkyl sulfonates (e.g., busulfan); (4) nitrosoureas (e.g., carmustine (BCNU) and streptozocin (streptozotocin); (5) triazenes (e.g., dacarbazine (DTIC; dimethyltriazenoimid-azolecarboxamide) and temozolomide).

In another embodiment, anticancer antimetabolite agents are employed in combination with cadherin antagonists. These may include, but are not limited to: (1) pyrimidine analogs (e.g., fluorouracil (5-fluorouracil; 5-FU) and floxuridine (fluoride-oxyuridine; FUdR); capecitabine, pemetrexed, cytarabine (cytosine arabinoside) and gemcitabine); (2) purine analogs and related inhibitors (e.g., mercaptopurine (6-mercaptopurine; 6-MP) and thioguanine) and/or (3) folic acid analogs (e.g., methotrexate).

Natural product-related anticancer agents may also be used in combination with cadherin antagonists according to the invention. These may include, but are not limited to: (1) vinca alkaloids (e.g., vinblastine (VLB) and vincristine); (2) taxanes (e.g., paclitaxel and docetaxel); (3) epipodophylltoxins (e.g., etoposide and teniposide); (4) camptothecins (e.g., topotecan and irinotecan); (5) antibiotics (e.g., dactinomycin (actinomycin D), daunorubicin (daunomycin; rubidomycin), doxorubicin, bleomycin, mitomycin (mitomycin C); and/or anthracycline agents (e.g., eiprubicin, idarubicin and liposomal doxorubicin). In a particular embodiment, the natural product-related anticancer agent is not a vinca alkaloid or paclitaxel.

In yet another embodiment, anticancer enzymes (e.g., L-asparaginase) and/or biological response modifiers or immunostimulators (e.g., interferon-alpha, interleukin-2 and other interleukins) may be used in combination with cadherin antagonists.

Still further anticancer agents which may be used in combination with cadherin antagonists may include, but are not limited to: (1) platinum-based anticancer agents such as platinum coordination complexes (e.g., cisplatin (cis-DDP), carboplatin and oxaliplatin); (2) anthracenediones (e.g., mitoxantrone); (3) methylhydrazine derivatives (e.g., procarbazine (N-methylhydrazine, MIH)); (4) adrenocortical suppressants (e.g., mitotane (o,p′-DD) and aminoglutethimide); (5) tyrosine kinase inhibitors (e.g., imatinib; erlotinib and gefitinib); and (6) multi-targeted kinase inhibitors (e.g., sunitinib; sorafanib and dasatinib).

Certain hormones and related antagonists may also be used according to the invention in combination with cadherin antagonists. These may include, but are not limited to: (1) adrenocorticosteriods (e.g., prednisone and prednisolone); (2) estrogens (e.g., diethylstilbestrol); (3) progestins (e.g., megestrol acetate); (4) aromatase inhibitors (e.g., exemestane and letrozole) and (5) antiestrogen (e.g., tamoxifen).

Anticancer antibodies are also useful in combination with cadherin antagonists. These may include, but are not limited to: (1) anti-angiogenesis antibodies (e.g., bevacizumab); (2) anti-CD20 antibodies (e.g., rituximab); (3) anti-epidermal growth factor receptor antibodies (e.g., cetuximab and panitumomab; and (4) radiolabelled antibodies (e.g., ¹³¹I-tositumomab).

In another embodiment, radiation therapy may be used in combination with cadherin antagonists including, for example, external beam therapy, implanted pellets, and other conventional radiation treatment methodologies.

### 3. Formulation, Dosing and Administration

It will be understood on the part of the skilled artisan, in view of this disclosure, that there exist a multitude of formulation, dosing and administration strategies that can be used according to the methods described herein to achieve an improved therapeutic benefit when using the methods described herein. Particular formulation components, dosing concentrations and/or administration schedules useful for a given combination of agents, while still achieving the therapeutic benefits described herein, may be routinely identified using skills and techniques known and established in the art. Accordingly, all such components, concentrations and/or schedules are considered within the spirit and scope of the present invention.

Cadherin antagonists and anticancer agents are administered to a subject or patient in need thereof in a manner appropriate to the cancer to be treated. The subject or patient can be essentially any cancer-bearing mammal such as a cancer-bearing dog, cat or human. Appropriate dosages, timing, duration and frequency of administration will be determined by such factors as the condition of the patient, the type and severity of the patient's disease and the method of administration. In general, an appropriate dosage and treatment regimen provides the agent(s) in an amount sufficient to achieve an improved therapeutic benefit, as described herein, relative to the separate components administered individually.

Optimal dosages for a given combination and a given indication may generally be determined using experimental models and/or clinical trials. In general, the use of the minimum dosage that is sufficient to provide effective therapy is preferred. Patients may generally be monitored for therapeutic effectiveness using assays suitable for the condition being treated or prevented, which will be familiar to those of ordinary skill in the art.

Suitable concentration/dosage ranges used for the anticancer agents and treatments described herein have been well defined, and the concentrations/dosages of the anticancer agents when used in the methods of the invention will generally be within these same established and accepted ranges. Typically, the concentration of an anticancer agent used in the methods of the invention will be at or below the maximum tolerated dose for the agent that is being used and/or at or below the typical dose when the agents are administered individually.

In one embodiment, the anticancer agent is melphalan and the agent is administered at a dose in the range of 1-30 mg/m² using a desired route/schedule. In another embodiment, mephalan is administered at about 4-8 mg daily PO according to standard schedules (e.g., 2-3 weeks). In another embodiment, mephalan is administered at about 10-20 mg/m² IV given according to standard schedules (e.g., 2 week intervals).

In another particular embodiment, melphalan is administered by an isolated limb infusion procedure and the dose of melphalan is about 7.5 mg/L limb volume infused for the lower extremity and about 10 mg/L limb volume infused for the upper extremity, with a maximum total dose of about 100 mg for lower extremity and about 50 mg for upper extremity. It will be understood that the dosage of melphalan can be corrected for ideal body weight using the following formula: Melphalan dose per Limb Volume×Ideal body weight÷Actual body weight=Corrected Melphalan Dose (Example: 90×120÷140=77 mg). The ideal body weight calculation program is included in the limb volume calculation program provided with the protocol. The limb volume can also be determined using a volume displacement water tank.

In another embodiment, the anticancer agent is 5-FU or an analog thereof and the agent is administered at a dose in the range of 100-1000 mg/m², using a desired route/schedule. In a particular embodiment, 5-FU is administered IV at about 500-1000 mg/m² according to standard schedules (e.g., weekly×4-8). In another embodiment, 5-FU is administered IV at about 500-1000 mg/m² in combination with about 500-1000 mg/m² of leucovorin according to standard schedules (e.g., weekly×4-8). In another embodiment, 5-FU is administered IV at about 250-750 mg/m² according to standard schedules (e.g., QD×5) repeated periodically (e.g., monthly). In another embodiment, 5-FU is administered IV at about 250-750 mg/m²/day infused according to standard schedules (e.g., for 21 days).

In another embodiment, the anticancer agent is cisplatin and the agent is administered at a dose in the range of about 25-300 mg/m², using a desired administration route/schedule. In a particular embodiment, cisplatin is administered IV at about 100-300 mg/m²/day according to standard schedules (e.g., ×5 days). In another embodiment, cisplatin is administered IV at about 50-200 mg/m² according to standard schedules (e.g., once every 4 weeks).

In another embodiment, the anticancer agent is paclitaxel and the agent is administered at a dose in the range of 25-300 mg/m2, using a desired administration route/schedule. In a particular embodiment, paclitaxel is administered IV at about 100-500 mg/m² infused according to standard schedules (e.g., 24 hour infusion), optionally in combination with G-CSF.

In one embodiment, the cadherin antagonist used according to the invention is a peptide-based antagonist, as discussed above, and is administered at a dose between about 10-2500 mg/m² using a desired administration route/schedule. In a more particular embodiment, the peptide-based antagonist is a cyclic peptide (e.g., comprising the sequence HAV, such as CHAVC (SEQ ID NO:1)) and is administered at a dose between about 400-900 mg/m². In another particular embodiment, the cyclic peptide is administered at a dose between about 500-700 mg/m².

The timing of administration for cadherin antagonists and anticancer agents or treatments may vary depending on the particular combination used, and specific timing is not critical provided that an improved therapeutic response is achieved in accordance with the present invention. Indeed, any of a variety of administration schedules and strategies may be identified and implemented by a skilled artisan for a given combination of agents while still achieving the objectives of this invention.

A cadherin antagonist is generally administered prior to administration of anticancer agent or treatment, for example about 7 days to about 1 hour prior to administration of anticancer agent or treatment. In another embodiment, a cadherin antagonist is administered after administration of anticancer agent or treatment, for example about 1 hour to about 3 weeks after administration of anticancer agent or treatment. Alternatively, a cadherin antagonist is administered approximately concurrent with administration of anticancer agent or treatment, for example within about 1 hour of administration of anticancer agent or treatment.

The route of administration for a cadherin antagonist and an anticancer agent or treatment may vary depending on the particular combination used, and specific delivery or administration routes are not critical provided that an improved therapeutic benefit is achieved in accordance with the present invention. Suitable delivery routes for the agents described herein are indeed well known and established and any such routes may be used in according with the invention. In many embodiments, cadherin antagonist will be administered systemically, preferably intravenously. Anticancer agents will generally be administered by their conventional and/or preferred routes and schedules of administration. Further, alternative administration schedules and strategies preferred for a given combination, and indication, may be identified and implemented by a skilled artisan using routine and standard methodologies.

In a particular embodiment, the anticancer agent is administered regionally (e.g., via intra-arterial chemotherapy delivery) and the cadherin antagonist is administered systemically. In another particular embodiment, cadherin antagonist is administered concurrently or prior to anticancer agent, e.g., about 1-4 hours prior to regional isolated limb infusion (ILI). In yet another particular embodiment, cadherin antagonist is administered at least about 0.5-4 hours prior to regional chemotherapy and administration of cadherin antagonist is optionally continued after chemotherapy stops.

In a more particular embodiment, the anticancer agent used in the methods of the invention is melphalan and the agent is administered via isolated limb infusion (ILI) at a dose in the range of 5-10 mg/liter leg limb volume to be infused and/or 5-15 mg/liter arm limb volume to be infused. Illustrative dose ranges for hyperthermic isolated limb perfusion (HILP), which is another form of regional intraarterial chemotherapy delivery, are about 5-15 mg/liter leg limb volume to be perfused and 13 mg/liter arm limb volume to be perfused.

Cadherin antagonists and anticancer agents may be present within a pharmaceutical formulation comprising at least one pharmaceutically acceptable carrier or excipient. A carrier or excipient is “pharmaceutically acceptable” in the sense of being compatible with the other ingredients of the formulation and being neither detrimental to the efficacy of the agents nor injurious to the patient. Cadherin antagonists may be present together in a single formulation for administration to a patient or, alternatively, may be present in separate formulations for administration at the same time or at different times.

Formulations may include, for example, those adapted for oral, rectal, nasal, topical (including buccal and sublingual), vaginal and parenteral (including subcutaneous, intramuscular, intravenous and intradermal) administration. The formulations may conveniently be presented in unit dosage form and may be prepared by any methods well known in the art of pharmacy. Such methods include the step of bringing into association the active ingredient with the carrier that constitutes one or more accessory ingredients. In general, the formulations are prepared by uniformly and intimately bringing into association the active ingredient with liquid carriers or finely divided solid carriers or both, and then if necessary shaping the product.

Formulations of the present invention adapted for oral administration may be presented as discrete units such as capsules, cachets or tablets each containing a predetermined amount of an active ingredient; as a powder or granules; as a solution or a suspension in an aqueous or non-aqueous liquid; or as an oil-in-water liquid emulsion or a water-in-oil liquid emulsion. An active ingredient may also be presented as a bolus, electuary or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder (e.g povidone, gelatin, hydroxypropylmethylcellulose), lubricant, inert diluent, preservative, disintegrant (eg. sodium starch glycollate, cross-linked povidone, cross-linked sodium caroxymethylcellulose) surface-active or dispersing agent. Molded tablets may be made by molding in a suitable machine a mixture of the powdered compound moistened with an inert liquid diluent. The tablets may optionally be coated or scored and may be formulated so as to provide controlled release of the active ingredient therein using, for example, hydroxypropylmethylcellulose in varying proportions to provide the desired release profile. Cadherin antagonist and anticancer agents can be formulated in tablets separately or combined within the same tablet.

Formulations for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier. Formulations for rectal administration may be presented as a suppository with a suitable base comprising for example cocoa butter or a salicylate. Formulation for vaginal administration may be presented as pessaries, tampons, creams, gels, pastes, foams or spray formulations containing in addition to the active ingredient such carriers as are known in the art to be appropriate.

Formulations for parenteral administration include aqueous and non-aqueous isotonic sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose sealed containers, for example, ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described.

The formulations described herein may further comprise other biologically active agents appropriate or desired for use in the intended application.

Cadherin antagonists may be present together in a single formulation for administration to a patient or, alternatively, may be present in separate formulations for administration at the same time or at different times.

The following Examples are offered by way of illustration and not by way of limitation.

### EXAMPLES

### Example 1

### In Vivo Combination Therapy of Melanoma Tumors Using an N-Cadherin Antagonist in Combination with Melphalan

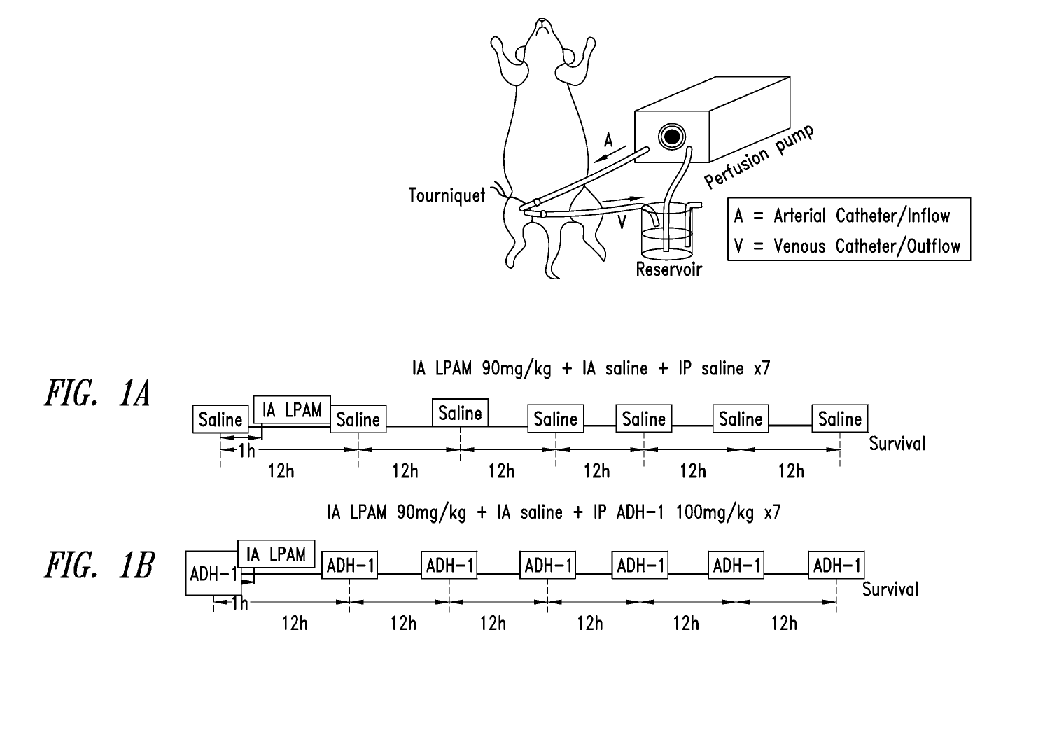

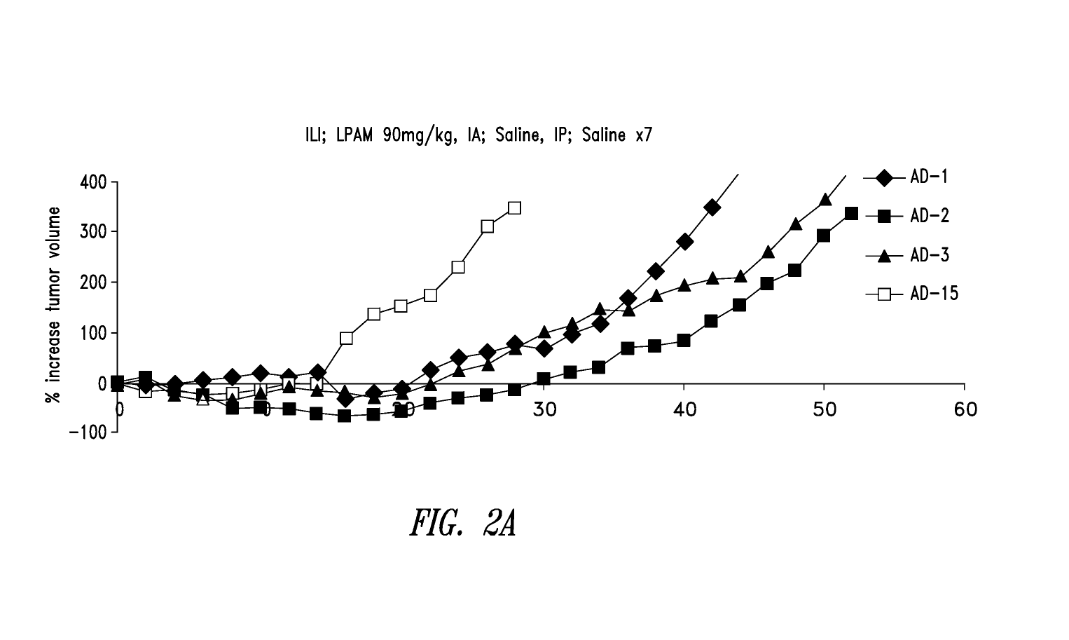

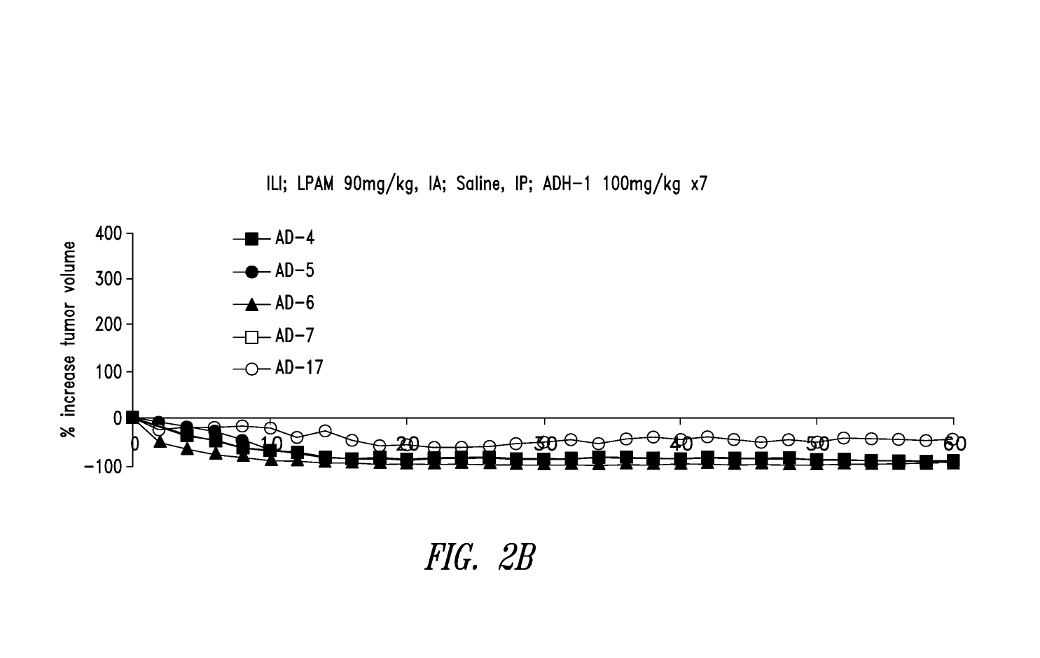

In a first set of experiments, nude rats were implanted with one of two different human melanoma xenografts (DM366 and DM738). When tumors reached 1 cm in size, the animals were treated systemically with 100 mg/kg ADH-1 (N—Ac-CHAVC-NH₂; SEQ ID NO: 1) or control prior to undergoing a 15 minute regional infusion (isolated limb infusion) with melphalan. Animals were followed for 60 days after treatment. A summary of the experimental protocol is provided in FIG. 1 and the results are presented graphically in FIG. 2. FIG. 2A shows tumor growth rates for melphalan alone infused into the hind limb. On day 60 there was a 439.4%+/−8.1 increase in tumor volume. FIG. 2B shows that if ADH-1 was administered systemically by IP injection 1 hr prior to the melphalan infusion, plus every 12 hours post for six consecutive administrations, there was essentially a 100% durable cure rate. The remaining very small but measurable mass was scare tissue as determined by H&E staining. There was no increase in toxicity, as measured by weight loss, when ADH-1 was used in combination with melphalan.

### Example 2

### In Vivo Combination Therapy of Resistant Melanoma Tumors Using an N-Cadherin Antagonist in Combination with Melphalan

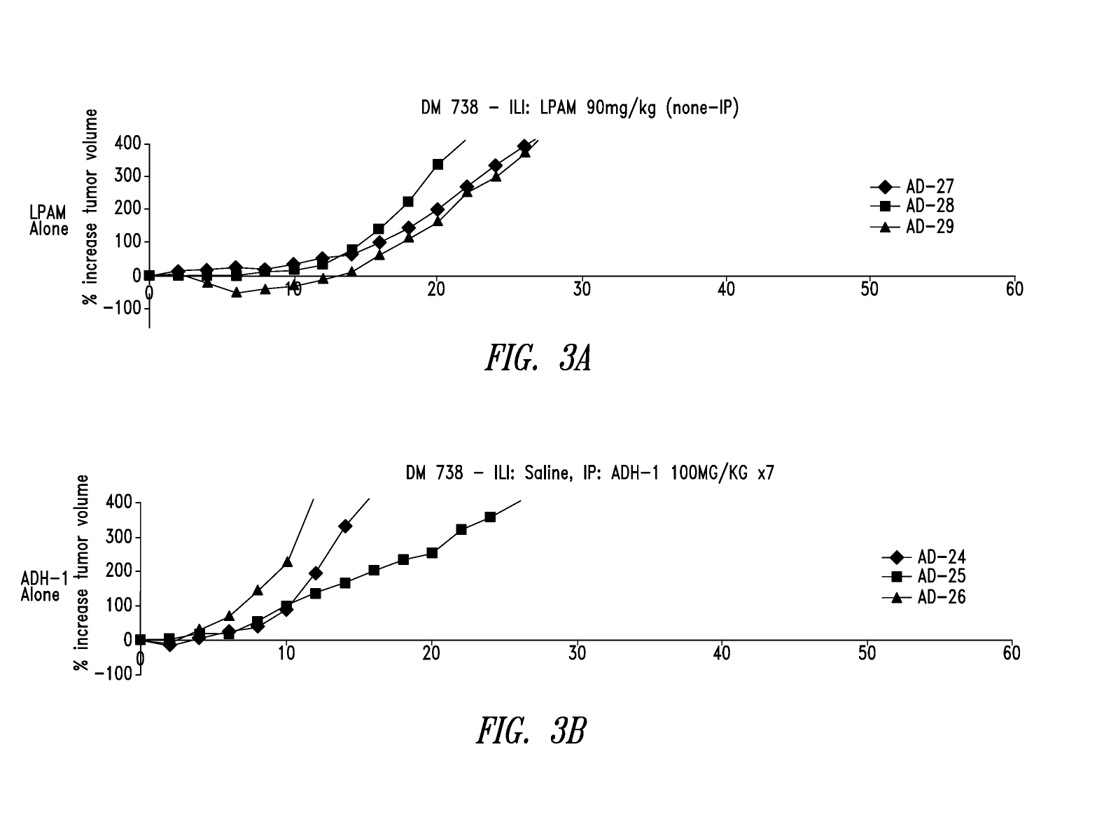

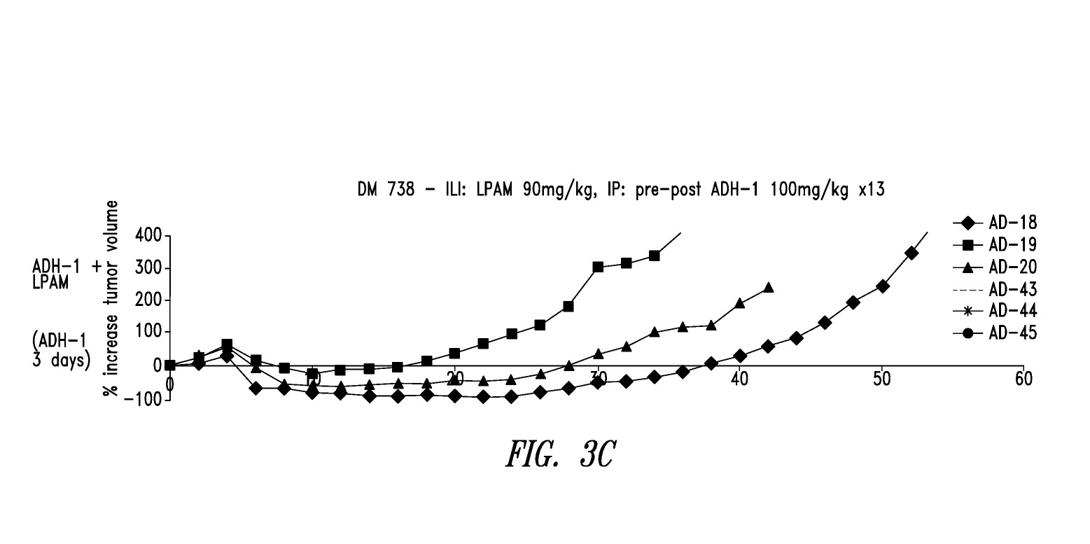

A second series of ILI experiments, essentially as described above, was conducted using the human melanoma cell line DM738. This cell line is resistant to melphalan. FIG. 3A shows that response of DM738 tumors to a single ILI of melphalan. There was no detectable anti-tumor response compared to vehicle control (not shown). FIG. 3B shows that there was no detectable anti-tumor response to systemically administered ADH-1 alone compared to melphalan alone or vehicle control. However, as shown in FIG. 3C, when ADH-1 was administered systemically and melphalan administered regionally in the isolated limb, there were significant antitumor effects without any increase in toxicity. The synergy between ADH-1 and melphalan converted a melphalan-resistant tumor to a melphalan-sensitive tumor.

### Example 3

### In Vivo Combination Therapy of Tumor Cell Line A375 Using an N-Cadherin Antagonist in Combination with Temozolomide (TMZ)

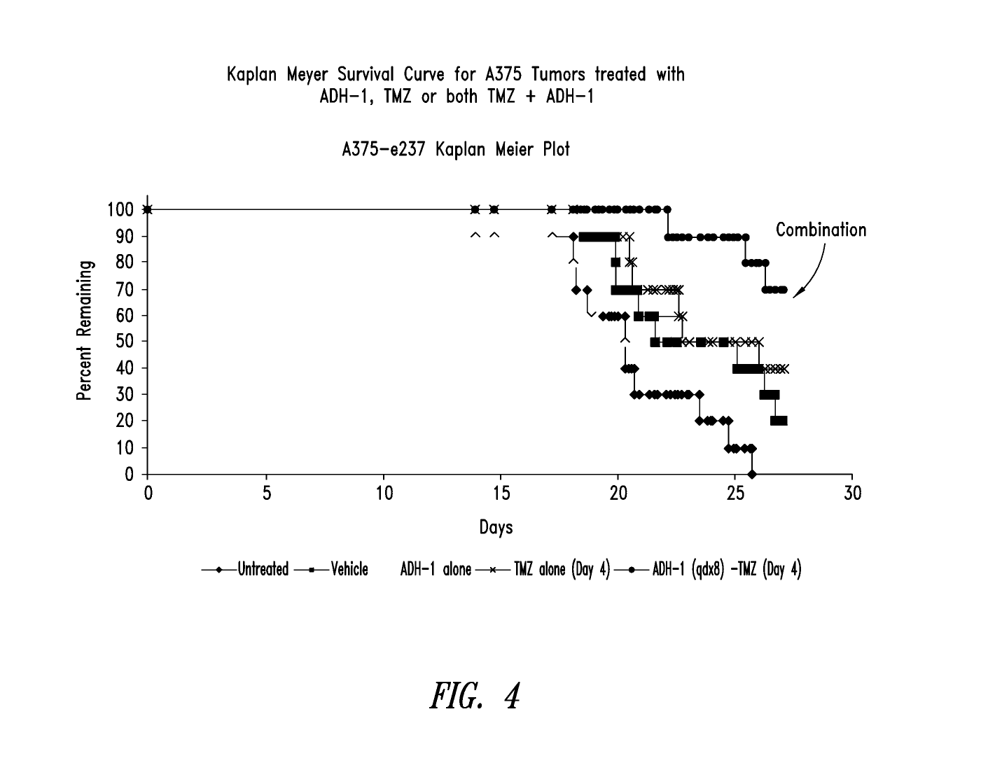

In this set of experiments, 100 mg/kg of ADH-1 was administered i.p. on a qdx8 schedule starting on Day 1. 100 mg/kg of TMZ was administered p.o. on a qdx5 schedule starting on Day 4. For the combination groups, TMZ was always administered 1 hour after the ADH-1 dose. A375 tumors were resistant to both TMZ and ADH-1 when administered separately. However, when administered in combination, there was a statistically significant increase in survival, as illustrated in the Kaplan Myer survival curve of FIG. 4.

### Example 4

### In Vivo Combination Therapy of Ovarian Tumor Cell Line A2780 Using an N-Cadherin Antagonist in Combination with Paclitaxel

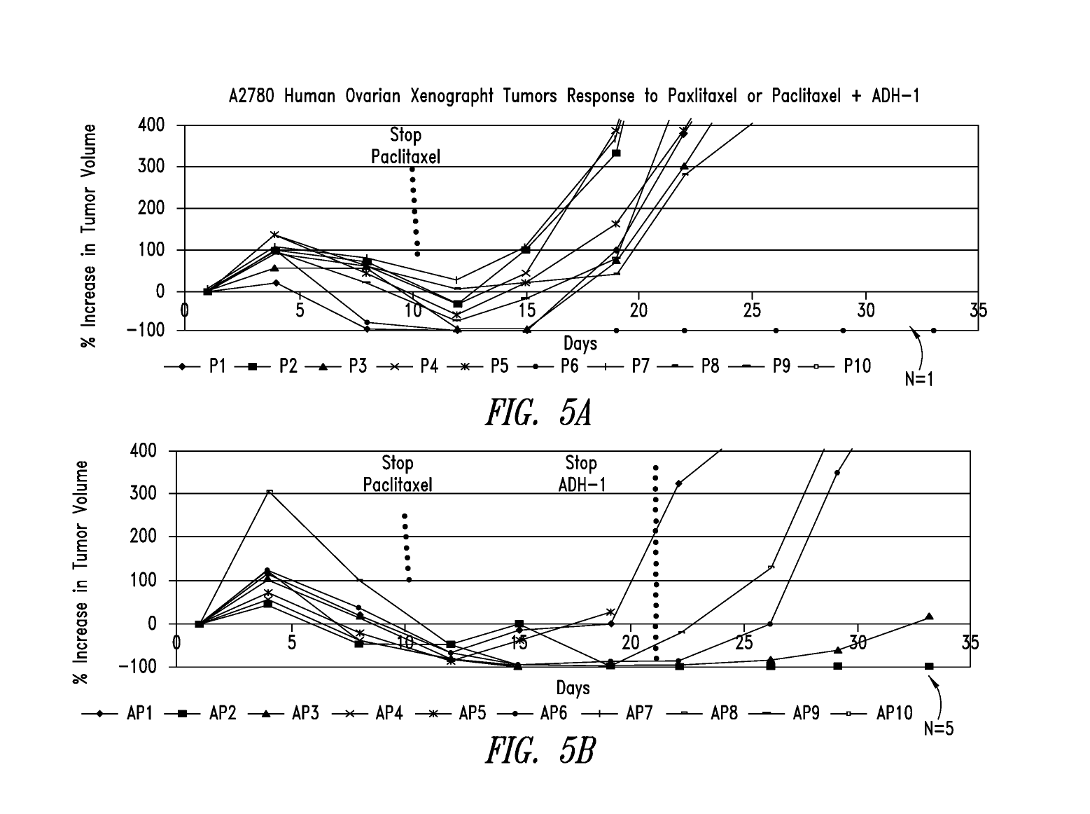

In this set of experiments, 100 mg/kg of ADH-1 was administered i.p. as a single dose on Day 1 and then b.i.d.x20 (i.p.) for the duration of the treatment period. 30 mg/kg paclitaxel was administered i.v. on a qodx5 schedule starting on Day 2. For the combination groups, paclitaxel was always administered 5 minutes after the morning ADH-1 dose. FIG. 5A illustrates that paclitaxel alone caused a transient delay in tumor growth with one complete cure out of 10 animals (10%). When ADH-1 used in combination with paclitaxel, there was a statistically significant delay in tumor outgrowth with 5 complete cures out of 10 animals (50%), as shown in FIG. 5B.

From the foregoing, it will be evident that although specific embodiments of the invention have been described herein for the purpose of illustrating the invention, various modifications may be made without deviating from the spirit and scope of the invention. Accordingly, the present invention is not limited except as by the appended claims.

1. A method for the treatment of a cancer comprising administering to a subject in need thereof at least one cadherin antagonist and at least one anticancer alkylating agent.

2. The method of claim 1, wherein the alkylating agent is selected from the group consisting of mechlorethamine, cyclophosphamide, ifosfamide, trofosfamide, melphalan (L-sarcolysin), chlorambucil, hexamethylmelamine, thiotepa, busulfan, carmustine (BCNU), streptozocin (streptozotocin), dacarbazine (DTIC; dimethyltriazenoimidazolecarboxamide) and temozolomide.

3. The method of claim 1, wherein the alkylating agent is melphalan.

4.-26. (canceled)

27. The method according to claim 1, wherein the cadherin antagonist is a peptide comprising the sequence HAV.

28. The method according to claim 27, wherein the cadherin antagonist is a cyclic peptide comprising the sequence HAV.

29. The method according to claim 28, wherein the cadherin antagonist is a cyclic peptide having the formula: 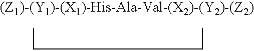
wherein X₁, and X₂ are optional, and if present, are independently selected from the group consisting of amino acid residues and combinations thereof in which the residues are linked by peptide bonds, and wherein X₁ and X₂ independently range in size from 0 to 10 residues, such that the sum of residues contained within X₁ and X₂ ranges from 1 to 12;
wherein Y₁ and Y₂ are independently selected from the group consisting of amino acid residues, and wherein a covalent bond is formed between residues Y₁ and Y₂; and
wherein Z₁ and Z₂ are optional, and if present, are independently selected from the group consisting of amino acid residues and combinations thereof in which the residues are linked by peptide bonds.

30. The method according to claim 29, wherein the cyclic peptide comprises a sequence selected from the group consisting of: N—Ac-CHAVC-NH₂ (SEQ ID NO:1). Another preferred cyclic peptide is N—Ac-CHAVC-Y—NH₂ (SEQ ID NO:2). Other cyclic peptides include, but are not limited to: N—Ac-CHAVDC-NH₂ (SEQ ID NO:3), N—Ac-CHAVDIC-NH₂ (SEQ ID NO:4), N—Ac-CHAVDINC-NH₂ (SEQ ID NO:5), N—Ac-CHAVDINGC-NH₂ (SEQ ID NO:6), N—Ac-CAHAVC-NH₂ (SEQ ID NO:7), N—Ac-CAHAVDC-NH₂ (SEQ ID NO:8), N—Ac-CAHAVDIC-NH₂ (SEQ ID NO:9), N—Ac-CRAHAVDC-NH₂ (SEQ ID NO:10), N—Ac-CLRAHAVC-NH₂ (SEQ ID NO:11), N—Ac-CLRAHAVDC-NH₂ (SEQ ID NO:12), N—Ac-CSHAVC-NH₂ (SEQ ID NO:13), N—Ac-CFSHAVC-NH₂ (SEQ ID NO:14), N—Ac-CLFSHAVC-NH₂ (SEQ ID NO:15), N—Ac-CHAVSC-NH₂ (SEQ ID NO:16), N—Ac-CSHAVSC-NH₂ (SEQ ID NO:17), N—Ac-CSHAVSSC-NH₂ (SEQ ID NO:18), N—Ac-CHAVSSC-NH₂ (SEQ ID NO:19), N—Ac-KHAVD-NH₂ (SEQ ID NO:20), N—Ac-DHAVK-NH₂ (SEQ ID NO:21), N—Ac-KHAVE-NH₂ (SEQ ID NO:22), N—Ac-AHAVDI-NH₂ (SEQ ID NO:23), N—Ac-SHAVDSS-NH₂ (SEQ ID NO:24), N—Ac-KSHAVSSD-NH₂ (SEQ ID NO:25), N—Ac-CHAVC-S—NH₂ (SEQ ID NO:26), N—Ac—S-CHAVC-NH₂ (SEQ ID NO:27), N—Ac-CHAVC-SS—NH₂ (SEQ ID NO:28), N—Ac—S-CHAVC-S—NH₂ (SEQ ID NO:29), N—Ac-CHAVC-T-NH₂ (SEQ ID NO:30), N—Ac-CHAVC-E-NH₂ (SEQ ID NO:31), N—Ac-CHAVC-D-NH₂ (SEQ ID NO:32), N—Ac-CHAVYC-NH₂ (SEQ ID NO:33), CH₃—SO₂—HN-CHAVC-Y—NH₂ (SEQ ID NO:34), CH₃—SO₂—HN-CHAVC-NH₂ (SEQ ID NO:35), HC(O)—NH-CHAVC-NH₂ (SEQ ID NO:36), N—Ac-CHAVPen-NH₂ (SEQ ID NO:37), N—Ac-PenHAVC-NH₂ (SEQ ID NO:38) and N—Ac-CHAVPC-NH₂ (SEQ ID NO:39).

31. The method according claim 29, wherein the cyclic peptide comprises the sequence N—Ac-CHAVC-NH₂ (SEQ ID NO:1).

32.-50. (canceled)
